(19) United States
(12) Patent Application Publication
(10) Pub. No.: US 2008/0311656 A1
(43) Pub. Date: Dec. 18, 2008

(54) Meiosis arrest in oocytes in vitro

(75) Inventors: Johan Smitz, Brussels (BE); Christian Grondahl, Vaerlose (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/228,349

(22) Filed: Aug. 12, 2008

Related U.S. Application Data
(63) Continuation of application No. 11/107,642, filed on Apr. 15, 2005, now abandoned.
Application No. 11/107,642 is a continuation of application No. PCT/DK2003/000703, filed on Oct. 15, 2003.

(30) Foreign Application Priority Data
Oct. 15, 2002 (DK) PA 2002 01581

(51) Int. Cl. C12N 5/08; C12N 5/06

(52) U.S. Cl. 435/366; 435/375

(57) ABSTRACT

The method for in vitro synchronisation of nuclear and cytoplasmatic maturation of GV oocytes from domestic animals or from primates can be improved if a phosphodiesterase type 3 inhibitor is added to the medium after collection of the oocytes and, thereafter, said phosphodiesterase type 3 inhibitor is removed to allow the nuclear maturation to proceed.

### CROSS REFERENCE TO RELATED APPLICATIONS

This application is a continuation of U.S. application Ser. No. 11/107,642 filed Apr. 15, 2005, which is a continuation of PCT Application PCT/DK2003/00703, published in English, designating the U.S., filed Oct. 15, 2003, claiming priority under 35 U.S.C. §119 from Danish Patent Application No. PA 2002 01581, filed Oct. 15, 2002.

### BACKGROUND OF THIS INVENTION

The present invention relates to a method for in vitro synchronisation of nuclear and cytoplasmatic maturation of germinal vesicle (hereinafter designated GV) oocytes from domestic animals or from primates. More particularly, the present invention relates to the aspects defined in the claims below.

Meiosis is the unique and ultimate event of germ cells on which sexual reproduction is based. Meiosis comprises two meiotic divisions. During the first division, exchange between maternal and paternal genes take place before the pairs of chromosomes are separated into the two daughter cells. These contain only half the number (1 n) of chromosomes and 2c DNA. The second meiotic division proceeds without a DNA synthesis. This division therefore results in the formation of the haploid germ cells with only 1c DNA.

The meiotic events are similar in the male and female germ cells, but the time schedule and the differentiation processes which lead to ova and to spermatozoa differ profoundly. All female germ cells enter the prophase of the first meiotic division early in life, often before birth, but all are arrested as oocytes later in the prophase (dictyate state) until ovulation after puberty. Thus, from early life, the female has a stock of oocytes which is drawn upon until the stock is exhausted. Meiosis in females is not completed until after fertilization, and results in only one ovum and two abortive polar bodies per germ cell. In contrast, only some of the male germ cells enter meiosis from puberty and leave a stem population of germ cells throughout life. Once initiated, meiosis in the male cell proceeds without significant delay and produces 4 spermatozoa.

Oocyte development during the follicular growth phase in vivo, is characterized by a prolonged arrest at prophase I until the pre-ovulatory surge of luteinizing hormone (hereinafter designated LH). During this period of oocyte development, intracytoplasmic changes related to synthesis and storage of RNA and protein translation occurs. These processes are essential to permit oocyte meiosis, fertilization and subsequent early embryo development. It is only at the time when the follicle reaches its maximal volume that the oocyte becomes developmentally competent. The LH rise releases meiotic arrest and the oocyte resumes the first meiotic division.

Prophase I arrest in mammalian oocytes is sustained by different mechanisms at distinct times of development. Growing oocytes, enclosed in primordial up to the early antral follicles, are arrested at prophase I and are germinal vesicle breakdown (hereinafter designated GVBD) incompetent because of a functional insufficiency. Up to this point, oocytes have not synthesised the cell cycle regulatory molecules essential for meiosis progression in sufficient quantities and/or these molecules are as yet not positioned correctly within the oocyte. In contrast, prophase I arrest in GVBD competent oocytes, enclosed in antral and preovulatory follicles, is sustained by interaction with the somatic cells, which provide appropriate levels of cAMP to the oocyte via gap junctions. Alternatively, the somatic compartment transfers inhibitory factors to the oocyte. In vivo, gonadotropins promote oocyte maturation indirectly via effects on granulosa cells, a process mediated predominantly via the cAMP system. A rise in follicular cAMP mediates LH action to induce oocyte maturation, and intraoocyte cAMP inhibits the process. This apparent contradiction can be explained by the fact that intraoocyte cAMP level decreases subsequently through the action of phosphodiesterases resulting in the resumption of meiosis. Although it has been suggested that meiotic resumption is caused by a drop off of cAMP in the oocyte due to an interruption of the gap junctions, there is evidence that GVBD occurs prior to any detectable ionic or metabolic uncoupling between these cells. Others have shown that the levels of intracellular cAMP do not decline during meiotic resumption. Although there are still some controversies on the role of cAMP for meiosis resumption, the concept that the second messenger cAMP plays an important role in meiosis arrest in different species is widely accepted.

Supplementing culture medium with compounds that maintain elevated cAMP can prevent spontaneous maturation. This can be achieved by several compounds: cAMP analogues such as dibutyryl cAMP (hereinafter designated dbcAMP), pharmacological agents that stimulate cAMP production via adenylate cyclase (forskolin) and inhibitors of the cAMP degrading isoenzymes phosphodiesterase (hereinafter designated PDE), such as 3-isobutyl-1-methylxanthine (IBMX).

In mammalians, resumption of meiosis occurs spontaneously when GVBD competent oocytes are liberated from their follicles in vitro. However, in the natural cycle, LH induces oocyte maturation (GVBD) in the follicle before ovulation occurs. Furthermore, it is known that in rodents antral and preovulatory follicles, about 4 mM of a PDE inhibitor (hypoxanthine) is continuously present. In humans, by the time of ovulation in vivo in natural cycles, the dominant antral follicle has been growing for about 14 days and has reached a diameter of approximately 22 mm. Several studies testified that oocytes aspirated from follicles not having reached a species specific minimal diameter, are incompetent to undergo nuclear maturation.

Certain phosphodiesterases (hereinafter designated PDEs) are responsible for the breakdown of cAMP, leading to a decrease of its intracellular levels. PDE consist of a large group of proteins in which at least eleven different families have been characterized. Examples of these families are type 3 PDEs and type 4 PDEs (hereinafter designated PDE3 and PDE4, respectively). Non-selective PDE inhibitors have been used to understand the role of cAMP in the resumption of meiosis. Suppression of cAMP catabolism by using different specific PDE inhibitors demonstrated the meiosis arresting action of these chemical compounds. PDE4 is mainly involved in the metabolisation of cAMP in granulosa cells. PDE 3 has been demonstrated to act directly in the oocyte without interfering with somatic cell functions. In the rat, expression of PDE3 was specific to the oocyte. The following statement can be found in the abstract in Developmental Biology 178 (1996), 393 et seq.: “In isolated oocytes, spontaneous GVBD was blocked by two inhibitors of type 3 PDE”. As appears from the publication, this statement applies to rats. It is stated in the abstract in J. Clin. Invest. 102 (1998), 532 et seq., that “inhibitors of phosphodiesterase 3 were used to block meiosis in ovulating oocytes in rodents. By this strategy, we [i.e., the authors of this paper] demonstrated that fertilization and pregnancy could be prevented”.

As appears from the above, cAMP signalling is a key factor during mammalian and amphibian oocyte maturation. In mammalians, germinal-vesicle stage (hereinafter designated GV) oocytes removed from immature antral follicles spontaneously resume meiosis in vitro but this process can be blocked in vivo and in vitro by certain compounds that maintain intraoocyte cAMP levels increased.

### SUMMARY OF THE INVENTION

One aspect of the present invention is to treat human infertility.

Another aspect of the present invention is to improve the maturation of human oocytes.

Another aspect of the present invention is to improve the synchrony of nuclear, cytoplasmic and/or membranous oocyte maturation.

Another aspect of the present invention is to improve the fertility of oocytes.

Another aspect of the present invention is to improve the rate of implantation of oocytes by human in vitro maturation and fertilisation.

Another aspect of the present invention is to diminish the incidence of human preembryos with chromosome abnormalities (aneuploidy).

Another aspect of the present invention is to improve the cleavage rate of human preembryos.

Another aspect of the present invention is to improve the quality of human preembryos.

Another aspect of this invention is to provide a method to better synchronize cytoplasmic and nuclear maturation for IVM.

Another aspect of this invention is to furnish a method permitting the IVM laboratory to operate within the normal workings hours of the day.

A further aspect of this invention is a method of increasing commodity of IVM practice by making the IVM independent from the time of oocyte cumulus complexes (hereinafter designated OCC) collection (i.e., the collection time can be uncoupled from meiosis progression and subsequent fertilization steps).

### BRIEF DESCRIPTION OF THE DRAWINGS

FIG. 1. Schematic diagram of the study.

FIG. 2. Illustrates the degree of cumulus-corona cells expansion originating from pre- or post-hCG treated patients at 0 hour followed by culture in control for 24 and 48 hours. On each column are marked the proportion of different COC types.

* denotes difference for types I and II COC from post-hCG (p<0.001).

** denotes difference for types II and IV COC from post-hCG (p<0.05).

FIG. 3. Illustrates the degree of cumulus-corona cells expansion originating from pre- or post-hCG treated patients at 0 hour followed by culture in DDMP for 24 and 48 hours. On each column are marked the proportion of different COC types.

* denotes difference for types I and II COC from post-hCG (p<0.05).

** denotes difference for type I COC from post-hCG (p<0.01).

*** denotes difference for types II and IV COC from post-hCG (p<0.05).

FIG. 4. Percentual proportion of nuclear maturation stages at 0, 24 and 48 hours culture of COCs collected from pre-hCG treated patients. N/A=oocytes not analyzed.

* denotes significant difference from controls (p<0.001) by χ² test.

FIG. 5. Percentual proportion of nuclear maturation stages at 0, 24 and 48 hours culture of COCs collected from post-hCG treated patients. N/A=oocytes not analyzed.

* denotes significant difference from controls (p<0.001) by χ² test.

FIG. 6. COC fixed immediately after retrieval. (a) Light microscopy of COC at time of retrieval. Note round cumulus-cells surrounding the oocyte. (×400). (b) Part of oocyte nucleus with intact nuclear membrane. Note the presence of the karyosphere surrounding the dark compact nucleolus. The cytoplasm surrounding the nucleus is covered with aggregates of mitochondria and vesicles of SER. (×3000). (c) Cortex of oocyte with clumps of mitochondria forming aggregates with SER (arrow). Note the areas devoid of organelles. Microvilli are projected from the oolemma to the inner cortex of ZP (*). Cortical granules (CG) dispersed throughout the cytoplasm and there is the start of formation of a single layer under the oolemma (arrowheads). (×3000). (d) Adherence between cumulus cells connected by gap junction (arrow). (×12000).

N=nucleolus; K=karyosphere; SER=smooth endoplasmic reticulum; MT=mitochondria;

FIG. 7. COC matured in vitro for 24 hours without inhibitor. (a) Light microscopy section with numerous elongated cumulus-cells. Note organelles dispersed throughout cytoplasm. (×400). (b) Cortex of polar body extruded oocyte with dispersion of organelles throughout cytoplasm. Note complexes of SER with peripheral mitochondria (*) (×3000). CG form one to two layers under oolemma (arrowhead). (c) Cumulus cells are elongated and retracted from the zona pellucida. The nuclei of the cells are positioned at the periphery at the opposite side of the elongation of the cytoplasm. Note the occasionally degenerating nucleus (arrow). (×1100). mc=microvilli.

FIG. 8. COC arrested in vitro for 24 hours by DDMP. (a) Light microscopy section with cumulus-cells starting to retract from zona but their nucleus is more centralised in the cell. Note organelles distributed in clumps in immature oocyte (GV not shown). (×400). (b) The nucleus is centrally located involved by an intact and smooth nuclear membrane (arrowhead). The nucleolus situated close to nuclear membrane is surrounded by a karyosphere. The mitochondriae are surrounding the GV intermingled with vesicles. (×1100). (c) and (d) The cortex of oocyte is devoid of organelles and mitochondriae are distributed forming clumps with SER. (d) Cumulus cells are retracted from zona. Lipid droplets are observed (arrowheads). (c=×3000; d=×1100).

GV=germinal vesicle; N=nucleolus; K=karyosphere; MT=mitochondria; ZP=zona pellucida.

FIG. 9. COC matured in vitro for 48 hours without inhibitor. (a) The first polar body is separated from the oocyte lying in a distended perivitelline space. Oocyte chromosomes appear as clumps of dense chromatin and lie on the equator of the metaphase spindle (arrow). Mitochondriae and SER are dispersed throughout the ooplasm, and one layer of CG appears beneath the oolemma (arrowhead). (×3000). (b) Cortex of PB extruded oocyte with two-three layers of CG and some vesicles of SER of which some are swollen (*). (×7000). PVS=perivitelline space; PB=polar body.

FIG. 10. COC arrested in vitro for 48 hours in the presence of DDMP. (a) Oocyte with centrally located nucleus with presence of karyosphere around the nucleoli. Note a still intact nuclear membrane (arrow). Organelles are intermingled with vesicles of SER, which are mainly localised around nucleus but start to dissociate from their aggregates. (×3000). (b) Oocyte at GV-stage has a more homogeneous distribution of organelles in the cytoplasm. Beneath the oolemma cortical granules are forming a single layer (arrows). (×1100). GV=germinal vesicle; N=nucleolus; K=karyosphere.

FIG. 11. COC arrested in vitro for 72 hours in presence of DDMP showing degenerative aspect. (a) Light microscopy section showing numerous increased vesiculation of SER around GV. (×1000). (b) Oocyte with signs of degeneration with increased vacuolisation of SER and mitochondria are also degenerated (arrowheads). Note lack of microvilli on oocyte cortex (arrow) and a more homogeneous zona (ZP). (×7000). SER=smooth endoplasmic reticulum. N=nucleolus; K=karyosphere.

FIG. 12. COC matured in vitro for 48 hours without inhibitor. Single sections of confocal images representing the microfilaments (actin) in M II oocyte. In (a) and (b) left and right are the same oocyte at different levels of the M II plate with chromosomal disarrangement.

FIG. 13. COC cultured for 30 h after arrest in vitro for 72 hours. Single sections of confocal images representing the microfilaments (actin) in M II oocyte. In (a) and (b) left and right are the same oocyte at different levels. The level of the first polar body F-actin displays a more predominant red staining than at the level of the metaphase plate.

### DETAILED DESCRIPTION OF THE INVENTION

### Definitions

MI is metaphase I. EGF is epidermal growth factor. GH is growth hormone. IVM is in vitro maturation. IVF is in vitro fertilisation.

Surprisingly, it was found that the blocking effect of PDE3INHs (which are defined below) could be reversed by washing out PDE3INHs and that a normal resumption of meiosis (normal time frame of meiosis progression and morphological aspect of the oocyte organelles) could be completed. It was demonstrated that normal offspring were born at a rate comparable to normal IVF.

For example, it has been found that favorable results are obtained by inducing an arrest in nuclear maturation concomitant with an extended in vitro culture period in a first culture medium (which is defined below). When cytoplasmic maturation is improved, the nuclear maturation is promoted by a second culture medium (which is defined below).

Furthermore, it has been possible to increase the normal yield of IVF/ICSI by retrieving OCC from the smaller follicles (<about 12 mm), which are normally not retrieved in a conventional treatment, but can, using this invention, now be used for IVM within a separate series of SOPs (i.e., standard operation procedures) and time schedule.

Surprisingly, when the PDE3INH is removed, meiosis will progress normally in most of the oocytes. For example, germinal vesicles (GVs) derived from follicles having a diameter from about 8 to about 12 mm will undergo normal chromatin re-modeling (surrounded nucleolus or karyosphere) during culture. Extension of the prophase arrest in vitro may result in cytoplasmic changes, but not in apparent nuclear changes.

By using a PDE3INH, interference with mechanisms upstream the oocyte pathway, more specifically the somatic cells cAMP-dependent pathways, can be avoided. The increased intra-oocyte cAMP results in an increase in type I isozyme protein kinase A (hereinafter designated PKA) activation, and subsequent phosphorylation of specific proteins, which are inhibitory to nuclear maturation.

As mentioned above, the present invention relates to a method for in vitro synchronisation of nuclear and cytoplasmatic maturation of GV oocytes as defined in the claims below. The method is a three step process. In the first step designated step (a), the oocytes are cultured in a first culture medium containing, inter alia, a PDE3INH and cytoplasmic maturation promoting substances or a co-culture system. In the second step designated step (b), a substantial part or all of the PDE3INH is removed from the oocytes. In the third step designated step (c), the oocytes are cultured in a second culture medium or a co-culture so that they become completed up to metaphase II to a desired frequency.

Preferably, the oocytes used in step (a) are collected from follicles not being too small. Usually, small oocytes from small follicles are to be cultured longer (in step (a)) than oocytes from larger follicles. Preferably, the oocytes used have a size which is at least about 70%, preferably at least 80%, more preferred at least about 90%, of the fully developed size of the oocytes of the species. For humans, it is preferred to use follicles having a diameter of about 4 mm to about 20 mm, preferably follicles having a diameter of about 6 mm to about 12 mm.

COCs can be retrieved from immature antral follicles in the follicular phase of the cycle before and after exposure in vivo to LH activity.

In a preferred embodiment of this invention, the oocytes used in step (a) are collected from follicles which were aspirated from the animal/human (cycling animal/human or not) before any hormone treatment that could effect either folliculogenesis and/or oogenesis). In another preferred embodiment of this invention, the hormone treatment of the woman from which the oocytes originates were performed with a steroid, a gonadotrophin, a GnRHanalogue, clomiphene citrate, tamoxiphen, an insulin sensitiser (e.g. metformin), an aromatase inhibitor or any of the preceding drugs alone or combined. In another preferred embodiment of this invention, the oocytes are collected from follicles which were aspirated from the animal after any hormone or drug treatment that effects upon folliculogenesis and/or oogenesis. In another preferred embodiment of this invention, the hormone or drug treatment is performed with a gonadotropin, a GnRHanalogue, clompihene citrate, tamoxiphen, LH, HCG, a steroid or a steroid-like substance, an LHRH analogue, an aromatase inhibitor, an insulin sensitizing agent, and any analogue or combination thereof. In another preferred embodiment of this invention, the hormone used is hCG, LH or any compound which might induce similar effects.

The medium used for culturing the oocytes is any medium containing the necessary nutrition components and which does not contain a substantial amount of compounds having an adverse influence of the cultivation of said oocytes. Such media are known to the skilled art worker. Furthermore, the skilled art worker is familiar with convenient culturing conditions such as temperature, osmolarity, humidity, time and the like.

Conveniently, the first medium contains as basis a bicarbonate buffer system (allowing a pH value of about 7.4 by incubation in a CO₂ incubator) and which has an osmolarity of about 285 mOsm per kilogram water. Culture is done at the adequate temperature for the species (about 37° C. for the human) and at a humidity of about 10%. Use can be made of several commercially available defined culture media or tissue culture media where a human serum albumin (which optionally is prepared by genetic engineering) is added as protein source. Examples of adequate media to use are DMEM or TC-199 supplemented inter alia by insulin (e.g., 1 ng/ml), transferrin (e.g., 5 microgr/ml), selenium (e.g., 5 ng/ml), 0.1% HSA, pyruvate, e.g., 23 microMolar, glutamine, e.g., 2 mM, and/or 17β-estradiol (hereinafter designated beta-E2).

In a preferred embodiment, the content of the PDE3INH in the first medium is in the range from about 0.1 to about 100 micromolar which, of course, can vary depending upon the potency of the specific PDE3INH used.

Conveniently, one or more cytoplasmatic promoting factors are added to the first medium. Specific examples of cytoplasmatic promoting factors are EGF (vide Fertil. Steril 55 (1991), 1000-1004; and Zygote 5 (1995), 345), GH (vide Mol. Reprod. Dev. 45 (1996), 372-377), gonadotropin combinations (vide Fertil. Steril. 52 (1989), 319-324; and Mol. Reprod. Dev. 45 (1996), 218-224), promoters of glutathione synthesis (vide Theriogenology 53 (2000), 761-771), meiosis activating sterols (vide Biol. Reprod. 58 (1998), 1297-1302), activin and inhibin (each alone or in combination) (vide Biol. Reprod. 58 (1998), 558-565; and Biol. Reprod. 56 (1997), 1559-1564), the analogues of the foregoing alone or in any combinations of the aforementioned compounds.

In step (a), the oocytes are, conveniently, incubated in a culture vessel which, conveniently, is covered by a minimal volume of oil compared to the volume of medium.

In step (a), the oocytes are to be culturing for a time period sufficient for cytoplasmatic maturation to progress or to allow for normal working hours during the IVF procedure.

One way of judging whether a time period is sufficient is to evaluate the outcome of the subsequent oocyte fertilization and embryo culture steps. The outcome of the subsequent oocyte fertilization step can be judged by any skilled art worker, e.g., number of 2 cells, the synchrony of nuclear, cytoplasmic and/or membranous oocyte maturation, the fertility of oocytes, the rate of implantation of oocytes by human in vitro maturation and fertilisation, the incidence of human preembryos with chromosome abnormalities (aneuploidy), the cleavage rate of human preembryos, and the quality of human preembryos. Outcome of fertilisation is usually checked by percent of 2 cells/inseminated oocyte. Outcome of embryo culture is judged by morphology of blastomeres (regularity or fragmentation), cleavage time intervals, blastocyst morphology and capacity to hatch.

Another way of judging whether a time period is sufficient for the cytoplasmatic maturation to progress is to use a validated molecular biology method (custom microarray technique) to access the absence or presence of selected markers expressed by the cumulus cell.

Herein the term PDE3INHs covers compounds being able to inhibit PDE3. PDE3s are specific enzymes present in 2 isoforms: PDE3A in myocardial, smooth muscle and in the oocyte and PDE3B functions in cells' hormonal regulation of lipolysis and glyconeogenesis (vide: J. Biol. Chem. 272 (1997), 6823-6826). PDE3A is the isotype of PDE3 which is present in the oocyte and regulates meiosis. The Type 3B (i.e., PDE3B) is present in other cells (fat cells) and is regulating lypolysis and neoglucogenesis.

The initial cloning of cAMP-specific PDEs (=cAMP-PDE: type 4) was followed by identifications of at least 25 different PDE forms in mammals. The PDEs were classified into seven distinct families (types) on the basis of their kinetic characteristics, substrate specificity, and regulation. A classification was proposed by in Mol. Pharmacol. 46 (1994), 399-405. A table on this issue is presented in Endocr. Rev. 16 (1995), 370-389.

More information on the PDEs can be found in Current Opinion in Cell Biol. 4 (1992), 233-240), Endocr. Rev. 16 (1995), 370-389, J. Biol. Chem. 268 (1993), 12925-12932, and Proc. Natl. Acad. Sci. USA 97 (2000), 12891-12895.

The human PDE3A and PDE3B genes have been cloned, and extensive studies have been performed to understand their patterns of expression (vide J. Biol. Chem. 272 (1997), 6823-6826).

PDE3B functions in the regulation of lipolysis and neoglucogenesis, while the PDE3A form is involved in the regulation of myocardial and smooth muscle contractility (vide J. Biol. Chem. 272 (1997), 6823-6826). The PDE3A isoform is also expressed in the rat and mouse oocyte.

The determination of whether a specific compound is a PDE31NH or not can be made by adding the compound in question to PDE3 and determine whether PDE3 is inactivated. For example, the concentration of cAMP in an oocyte goes up if PDE3 is inhibited. More specifically, this can, for example, be made by determining whether the concentration of cAMP in an oocyte goes up if the compound to be tested is added. In that case, oocyte maturation is prevented (i.e., the GV block of meiotically competent oocytes is maintained) but there is no effect on the somatic cells (i.e. there is no increased cAMP-dependent steroid production increase). An example of such an experiment (i.e. to establish the specificity and dose dependent ability of PDE3, but not PDE4, inhibitors to block the spontaneous maturation of meiosis) was described in Human Reproduction 17 (2002), 2019-2084.

Examples of specific PDE31NHs are milrinone, cilostamide, fenoximone and compounds of the general formula I mentioned in European patent application having publication number 0 350 990 (hereinafter designated EP 350,990), for example, 4,5-dihydro-6-(5,6-dimethoxybenzo[b]thien-2-yl)-5-methyl-3(2H)-pyridazinone or 4,5-dihydro-6-(5,6-dimethoxybenzo[b]thiophene-2-yl)-5-methyl-3(2H)-pyridazinone (hereinafter designated DDMP (alternatively designated ORG 9935)) and having the formula:

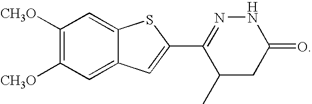

The concentration of the DDMP in the first medium is conveniently in the range from about 1 μM to about 100 μM.

Examples of domestic animals are dogs, cats, cows, pigs, horses, and sheep. Examples of primates are monkeys and humans, preferably humans.

In a preferred embodiment of this invention, the oocytes used for culturing in the first medium are oocytes which have been stored in an oocyte collection medium being a CO₂-independent medium containing PDE31NH.

In a preferred embodiment of this invention, the oocyte has been placed in said oocyte collection medium not later than 4 hours, preferably not later than 2 hours, even more preferred not later than 1 hour, and even more preferred not later than ½ an hour, after said oocyte has been removed from the woman.

In a preferred embodiment of this invention, the culturing according to step (a) takes place for a time period sufficient for cytoplasmatic maturation to be improved. In another preferred embodiment of this invention, the culturing according to step (a) takes place for a time period sufficient for cytoplasmatic maturation to be completed.

In a preferred embodiment of this invention, the oocytes cultured in step (a) are GV oocytes.

In a preferred embodiment of this invention, the first culture medium contains EGF, GH, gonadotropin combinations, promoters of glutathione synthesis, meiosis activating compounds, combinations of activin and inhibin, the analogues of the foregoing or combinations of the aforementioned compounds.

In a preferred embodiment of this invention, the PDE31NH is a compound covered by claim 1 in EP 350,990, preferably DDMP.

In a preferred embodiment of this invention, the culturing time in step (a) is at least about 4 hours, preferably at least about 8 hours, more preferred at least about 12 hours, even more preferred at least about 24 hours, more preferred at least about 48 hours and even more preferred at least about 72 hours.

In a preferred embodiment of this invention, the first culture medium is minimally covered by a thin layer of an oil so that the ratio between the oily phase and the aqueous phase is less that about 2:10 (vol/vol), preferably less that about 1:10 (vol/vol). In another preferred embodiment of this invention, the oocyte collection medium is covered by oil.

In a preferred embodiment of this invention, there are cumulus cells around the oocyte used in step (a).

Conveniently, the second medium contains the same basis components as the first medium (see above) to which is added HCG, LH or MAS, analogues of these compounds alone or in any combination.

In a preferred embodiment of this invention, the oocytes of step (a) are washed in order to remove PDE31NH or decrease the amount thereof, so that any PDE31NH present has no substantial effect on the arresting of the oocytes.

In a preferred embodiment of this invention, the washing of the oocytes from step (a) is performed so that the concentration of PDE31NH after the washing out of PDE31NH in the second medium is less than about 10%, preferably less than about 5%, more preferred less than about 1% of the concentration of PDE31NH in the first medium.

In the third step, the oocytes cultured in the second medium can be covered by an amount of oil.

In a preferred embodiment of this invention, there are no or substantially no cumulus cells around the oocyte in step (c).

In a preferred embodiment of this invention, the nuclear maturation is stimulated by adding HCG and/or EGF and/or a MAS compound to the second culture medium in step (c). In case there are no cumulus cells present around the oocyte (most times after a long culture period cumulus drops off the oocyte), HCG or EGF cannot effect on oocyte. However, MAS compounds can in such cases.

In a preferred embodiment of this invention, the desired frequency in step (c) for nuclear maturation to be completed up to metaphase II is at least about 30%, more preferred at least about 50%, even more preferred at least about 70%, and preferably at least about 90%.

In a preferred embodiment of this invention, after step (c), the oocytes are fertilized with sperm, preferably using ICSI.

The present invention makes it possible to steer away from working in the weekends or in the evening or night.

The nuclear maturation has been completed when there is a first polar body extrusion. This can, for example, be determined microscopically.

Metaphase II is the stage where the first polar body is present in perivitteline space.

In a preferred embodiment of this invention, the oocytes used in step (a) have been stored in an oocyte collection medium which, conveniently, is a CO₂-independent medium containing PDE31NH. For example, it could be a HEPES buffered medium or a PhoSphate medium (for example Leibovits medium). The oocytes are stored in this oocyte collection medium as soon as they come out of the follicle. The reason for this is that, otherwise, the oocytes might be collected in distant places (satellite collection centres and field conditions and are then transported to the IVF/ICSI laboratories). In the latter case, if time goes over, there might not be a sufficient inhibition of oocyte maturation present. The use of such an oocyte collection medium is to avoid that oocytes start to resume meiosis, especially if they are of the more denuded-type from cumulus cells.

All references, including publications, patent applications, and patents, cited herein are hereby incorporated by reference to the same extent as if each reference were individually and specifically indicated to be incorporated by reference and were set forth in its entirety herein (to the maximum extent permitted by law).

Any combination of the above-described elements in all possible variations thereof is encompassed by the invention unless otherwise indicated herein or otherwise clearly contradicted by context.

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising,” “having,” “including,” and “containing” are to be construed as open-ended terms (i.e., meaning “including, but not limited to,”) unless otherwise noted and should be read as encompassing the phrases “consisting”, “substantially comprised of,” and “consisting essentially of” (e.g., where a disclosure of a composition “comprising” a particular ingredient is made, it should be understood that the invention also provides an otherwise identical composition characterized by, in relevant part, consisting essentially of the ingredient and (independently) a composition consisting solely of the ingredient).

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

Preferred embodiments of this invention are described herein. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law.

This invention is further illustrated in the following experiments which, however, are not to be construed as limiting the scope of protection.

The features disclosed in the foregoing description and in the following may, both separately and in any combination thereof, be material for realizing this invention in diverse forms thereof.

### EXAMPLE 1

### Material and Methods

### Collection of Immature Oocytes

Patients included in this study were undergoing fertility treatment and were primed with variable doses of gonadotropins while being carefully monitored using serum hormone and vaginal ultrasound monitoring (Human Reproduction 7 (1992), 49 et seq.). Collection of oocytes cumulus complexes (COCs) from different follicle sizes was performed as follows: 1°) during laparoscopic surgery, 2°) aspirated from patients with stimulated ovaries undergoing follicle reduction before intrauterine insemination (to avoid multiple gestation due to multiple ovulation) (Albano et al, 2001) or 3°) during ovum pick up after having aspirated the larger follicles (>15 mm) destinated for IVF treatment (Human Reproduction 7 (1992), 49 et seq.). Basically, these aspirations yielded two types of COC: a first group that had never been exposed to LH activity (pre-hCG) and a second group of follicles 36 h after an exposure to hCG (injected) or endogenous LH (spontaneous LH peak). When possible, follicle diameters were recorded by vaginal ultrasound measurement. COCs used in this study were retrieved from follicles with diameters between 6 and 14 mm.

Human oocytes aspiration was performed by ultrasound-guided transvaginal follicle aspiration using a single lumen needle (Cook, K-OPS-1230-VUB, Switzerland AG) after paracervical infiltration with a local anesthetic. The aspiration pressure used was reduced from the usual 100 mmHg for recovery of mature oocytes to 60 mmHg. Follicles were flushed using HEPES-buffered Earle's medium at least three times.

### Classification of Cumulus Oocyte Complexes (COCs)

The COCs were evaluated immediately after aspiration and after 24 hours and 48 hours culture and classified as follows: type (I), compact mass of 3-5 layers of granulosa cells; type (II), expanded distal layers of granulosa cells (cumulus), but a compact proximal granulosa cell layer; type (III), expanded distal (cumulus) and proximal (corona cells) layers of granulosa cells; and type (IV) partially denuded oocytes due to expanded granulosa cells.

### Culture of COCs

At the time of oocyte retrieval only the COCs classified as I or II were placed in culture.

Twenty-four hours later, oocyte stages were recorded and oocytes showing a clear polar body extrusion were removed from culture. After 48 hours, all oocytes were assessed and GV, GVBD and polar body extruded ones were recorded.

The basal medium used for oocyte culture was D-MEM (D-5280, Life Technologies, Merelbeke, Belgium) with 2 mM Glutamax-1, NaHCO₃ (2 g/L) supplemented with 10 mIU/ml FSH (Gonal-F®, kindly donated by Ares Serono, Geneva, Switzerland), 100 ng/ml IGF-I (R&D, UK), 5 ng/ml insulin, 5 ng/ml selenium, 5 μg/ml transferrin (Life technologies, Merelbeke, Belgium) and 0.1% human serum albumin (CAF-DCF, Brussels, Belgium). 17β-E₂ (Sigma, Bornem, Belgium) was diluted in ethanol and added to oil to reach a final concentration of 1 μg/ml in the 10 μl medium droplets underneath (this concentration was measured using specific radioimmunoassay). Microdroplets of 10 μl medium, were covered with 2 ml of oil. A refreshment of 511 conditioned medium was performed each 24 hours. All cultures were carried out at 37° C. in humidified atmosphere in an incubator gassed with 5% CO₂ in air.

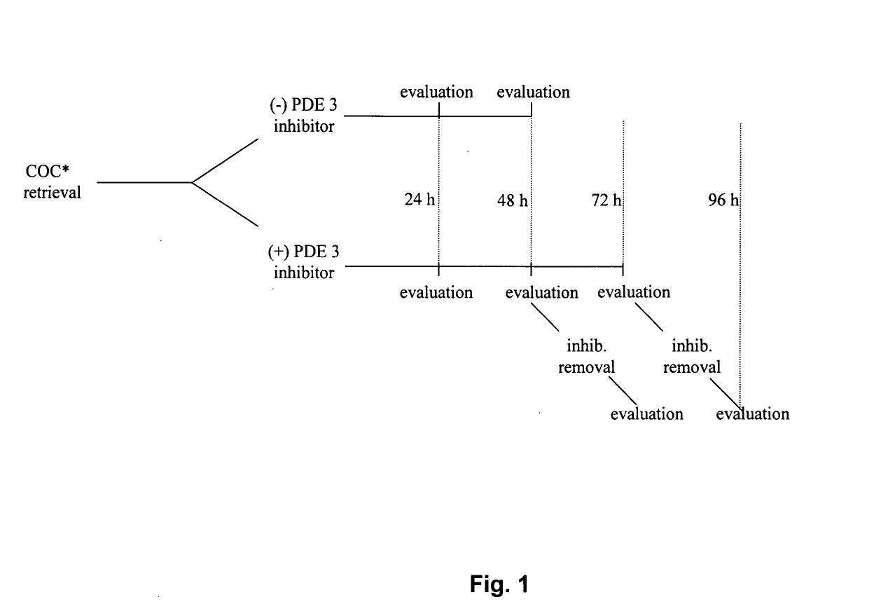

Schematic diagram of the study is shown in FIG. 1.

### Oocyte Inhibition and Reversal of Meiosis

DDMP was added at a 10 μM final concentration (0.1% DMSO). Directly after aspiration, single COCs were washed in flushing medium (D-MEM with HEPES) and placed into 10111 droplets under oil. COCs were randomised over two culture conditions: with and without DDMP. Oocytes cultured with DDMP for 24 hours, 48 hours and 72 hours were studied for reversal of meiosis arrest by washing out the inhibitor. Reversal of PDE 3 inhibition was performed by mechanical denudation of the remaining somatic cells and placing the oocytes in the conditioned medium for an additional 24-30 h.

### Oocyte Preparation for Electron Microscopy (Hereinafter Designated EM)

During evaluation at 24 hours and 48 hours culture, part of the oocytes with and without DDMP were fixed for EM analysis.

A total of 48 oocytes (4 at time of retrieval, 21 after 24 hours, 20 after 48 hours and 3 after 72 hours culture) were fixed in glutaraldehyde 2.5% in cacodylate buffer at pH 7.3 for at least 2 hours at 4° C. at time 0 h, 24 hours and 48 hours of maturation. All oocytes were embedded individually. Afterwards, they were postfixed in OsO₄ 1% in H₂O for 30 min, stained with uranyl acetate 2% in veronal buffer for 60 min, dehydrated through graded alcohols, embedded in SPURR (Taab; Bodson, Liège, Belgium) and left to polymerize overnight at 70° C.

Semithin sections of 0.5 μm and ultrathin sections of 75 nm were made at approximately 30 levels (range 15-35). The semithin sections were mounted on glass slides and stained with toluidine blue for light microscopical guidance. The ultrathin sections were transferred on wide single slot copper grids (Agar Scientific LTD, Stansted, UK), coated with a polyvinyl formal film (Formvar, Laborimpex, Brussels, Belgium), stained with lead citrate and examined using a Zeiss transmission electron microscope type EM 9S2.

### Oocyte Preparation for Confocal Laser Microscopy

For localization of microfilaments, 39 polar body extruded oocytes (25 oocytes 48 hours after maturation in control medium and 14 oocytes after inhibitor removal) were fixed with 2.5% glutaraldehyde in cacodylate buffer for at least 2 hours, washed with PBS/1% BSA, placed into 0.1% Triton X-100 for 5 min and rinsed again with PBS. Afterwards, oocytes were placed into Texas red-labeled phalloidin staining f-actin diluted 1:40 PBS for f-actin and Pico green 1:2000 PBS for DNA staining.

### Statistical Analysis

Evaluation of cumulus morphology expansion in culture related to follicle treatment, resumption of maturation, kinetics of GVBD and polar body extrusion were analysed using the χ²test and Fisher's exact test for comparison between control and DDMP treated groups. Statistical significance was considered when P<0.05.

### Results

### Oocyte Recovery Rates in the Different Groups

A total of 139 follicles were aspirated from 30 patients before any hCG administration. The follicles between 6 and 12 mm (45.3%) yielded an oocyte recovery rate of 80.9% (mean±SD of oocytes per patient: 3.2±2.2; range 1-9). The aspirated follicles larger than 12 mm (54.7%) yielded an oocyte recovery rate of 35.5% (mean±SD of oocytes per patient: 1.7±0.9; range 1-4).

A total of 128 follicles between 6 and 12 mm were aspirated from 27 patients at 36 hours after hCG administration. These yielded an oocyte recovery of 50.0% (mean±SD of oocyte per patient: 2.4±1.5; range 1-6). A total of 54 follicles between 13 and 15 mm were aspirated from 9 patients with an endogenous LH peak a day before follicle aspiration. These yielded an oocyte recovery of 37.0% (mean±SD of oocyte per patient: 2.2±0.8; range 2-4).

### Morphological Characteristics of COCS

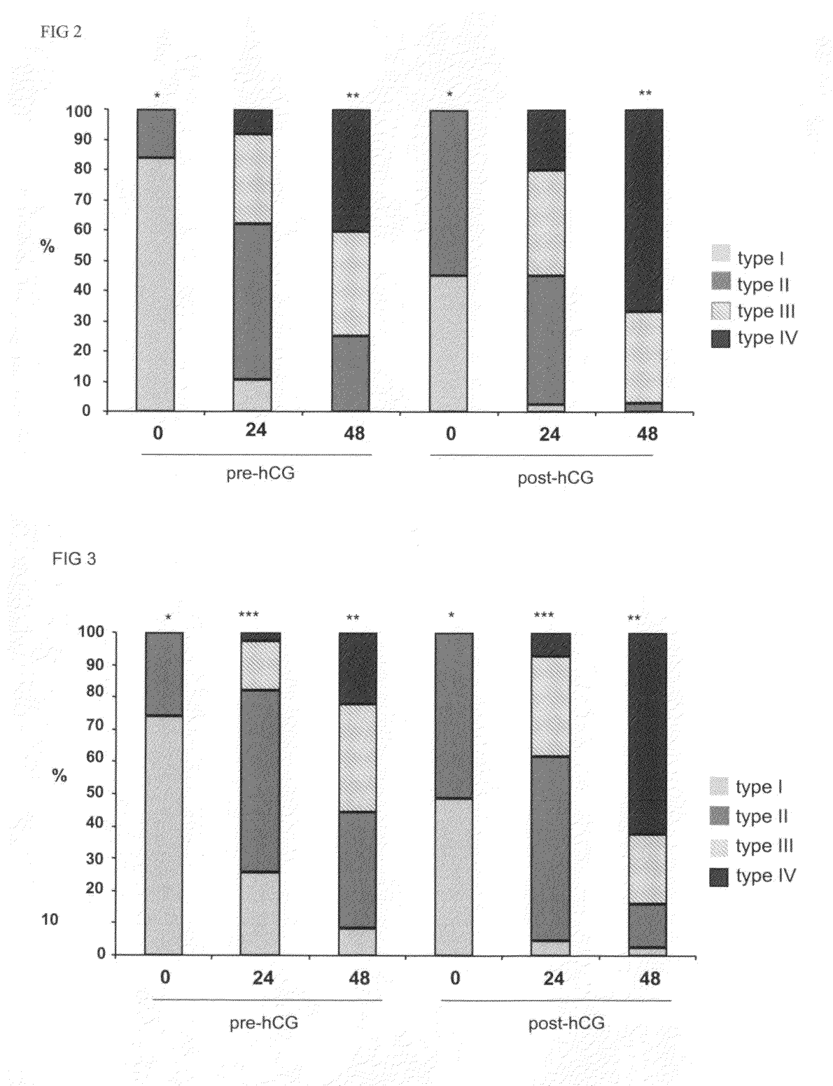

### Cumulus Cell Expansion at the Moment of Retrieval and During Culture in Controls (FIG. 2)

### COC Retrieved Pre-hCG Administration

At retrieval there was a high proportion of COCs with compact cumulus morphology (type I) (83.8%). After 24 hours, in controls the proportion of types I COCs decreased to 10.8%. At this time, 51.4% and 29.7% COCs were classified as type II and III, respectively. At 48 hours culture, 40% of oocytes had lost their connections with the cumulus cells (type IV).

### COC Retrieved Post-hCG Administration

This group comprises the patients in whom hCG was administered for 36 hours (normal IVF/ICSI procedure) and those patients who had an endogenous LH surge. The COCs retrieved from the post-hCG patients presented a significantly lower proportion of type I COCs compared to pre-hCG patients (45.0%) (p<0.01). Twenty-four hours later, almost all COCs presented an expanded cumulus (42.5% of type II and 35.0% type III) and 20.0% had already lost the connections cells-oocyte. Finally at 48 hours culture, a significantly higher proportion of oocytes had lost their connections with the cells compared to the cultured ones from the pre-hCG group (66.7%) (p<0.05).

### Cumulus Cell Expansion in the Presence of the DDMP (FIG. 3)

### COC Retrieved Pre-hCG Administration

From the retrieved COC from pre-hCG patients, 74.4% were classified type I. After 24 hours, the proportion of type I decreased to 25.6%. At this time, 56.4% and 15.4% COCs were classified type II and III, respectively. Finally at 48 hours culture, 22.2% of oocytes had lost their connections with the cumulus cells.

### COC Retrieved Post-hCG Administration

The proportion of type I COCs retrieved from the post-hCG patients was significantly lower (48.8%) compared to those retrieved from pre-hCG patients (p<0.05). Twenty-four hours later, only 4.7% from post-hCG cumuluses remained type I. This constitutes a significantly lower proportion than the pre-hCG cumulus (p<0.05). There were 57.1% of type II and 30.9% type III cumuluses (not significantly different from pre-hCG). After 24 hours, a similar proportion of oocytes as in the pre-hCG group had lost connection with cumulus cells (7.1%). Finally at 48 hours culture, 62.1% of oocytes had lost connections with the somatic cells, this is higher than COCs retrieved from the pre-hCG group (p<0.05).

The expansion and mucification pattern of COC before and during culture was not modified by the presence of PDE 3 in medium.

### Presence of DDMP in Culture: Timed Analysis of Nuclear Maturation Evaluation at 24 Hours of Culture

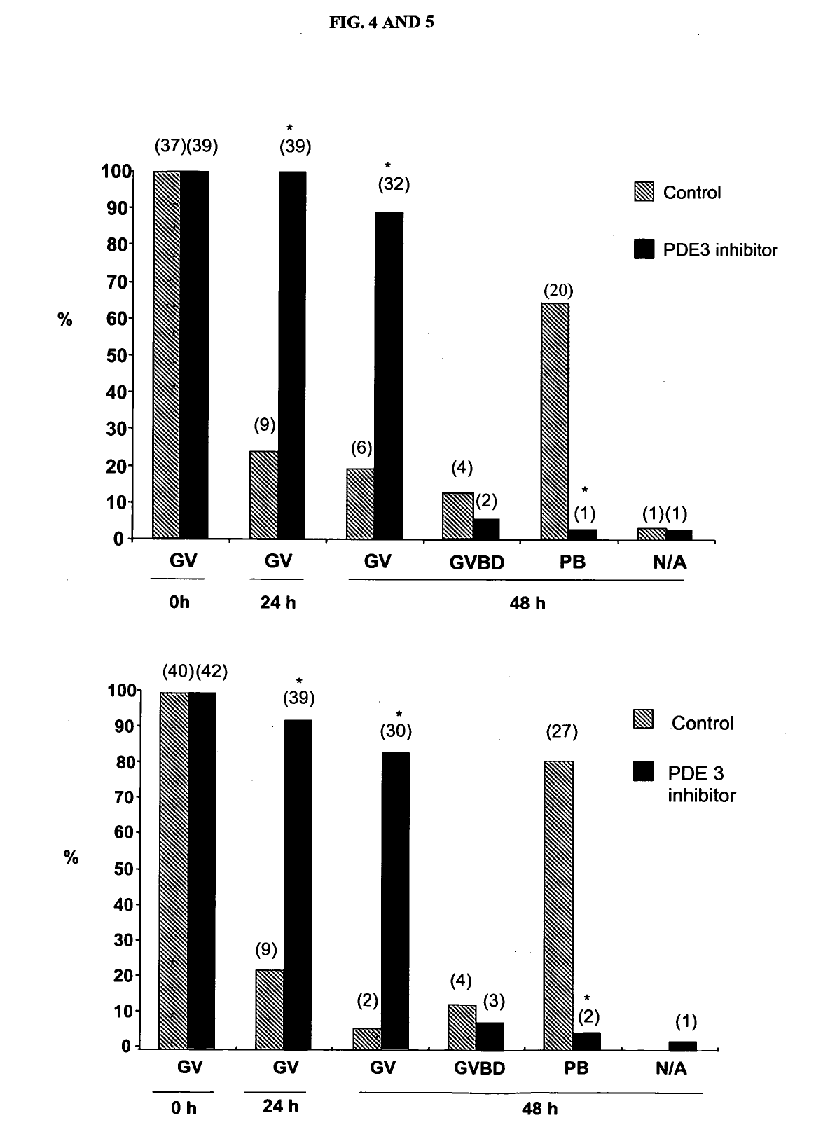

### COC Retrieved Pre-hCG Administration (FIG. 4)

A total of 39 COCs at GV-stage were cultured in presence of DDMP and 37 COC without (=control). After 24 hours culture, all oocytes remained at GV-stage when DDMP was present in the medium in contrast to only 24.3% in control medium (p<0.001). In controls, four oocytes (10.8%) had a polar body extruded. A large proportion of oocytes (64.9%) did not show clear presence of a polar body into the perivitelline space (not shown in Fig.).

### COCs Retrieved Post-hCG Administration (FIG. 5)

A total of 42 immature COCs were cultured in the presence of DDMP and 40 COCs in control. COCs retrieved from exogenous (hCG) and endogenous (spontaneous) LH peaks were grouped in the same tables and Figures. After 24 hours culture, 92.9% of oocytes remained at GV-stage in DDMP medium while only 22.5% in controls (p<0.001). Of those oocytes, which did not arrest at GV-stage despite DDMP, only 2.4% extruded a visible polar body (PB) (not shown in graph). In controls, 10.0% had extruded a polar body (PB). The proportion of oocytes in which it was impossible to visualize the nucleus to be sure of the presence of PB was 67.5% in controls.

### Evaluation at 48 Hours of Culture

### COCs Retrieved Pre-hCG Administration (FIG. 4)

COCs in which the nuclear stage was not clearly visualized were mechanically denuded. Those that were fixed for EM analysis had their maturational stage determined afterwards (on the semithin sections). Oocytes cultured in presence of the DDMP, remained for 88.9% at the GV-stage in contrast to only 19.3% in controls (p<0.001). In the presence of DDMP, only 5.5% of oocytes were GVBD, 2.8% were PB and in 2.8% the stage could not be determined. In controls, 12.9% were GVBD, significantly more oocytes had a PB extruded (64.5%) (p<0.001) and 3.2% had a non determinable stage. The relation between follicle diameter recorded and number of polar body extruded oocytes after culture is shown in Table 2

TABLE 2
Number of oocytes reaching normal polar body extrusion after 24 or 48
hours of in vitro culture in control medium. Data grouped according to
follicle size at retrieval and treatment by hCG or not

| Follicle | | Number of | Number of PB extruded | |
|---|---|---|---|---|
| size at | Patient | oocytes at time | oocytes between 24-48 hours | |
| retrieval | treatment | of retrieval | culture; (% of total) | |
| >12 mm | Pre-hCG | 13 | 11 | 84.6 |
| | Post-hCG | 11 | 11 | 100.0 |
| ≦12 mm | Pre-hCG | 24 | 13 | 54.1 |
| | Post-hCG | 29 | 21 | 72.4 |

### COCs Retrieved Post-hCG Administration (FIG. 5)

COCs cultured in presence of DDMP showed a significant greater proportion of oocytes at the GV-stage (83.3%) compared to controls (6.0%) (p<0.001). In DDMP, 8.3% of oocytes were GVBD, only 5.6% were PB and 2.8% had a not determined stage. In controls, 12.1% were GVBD and a significant greater proportion of oocytes had a PB extruded (81.8%) (p<0.001). The relation between polar body extruded oocytes and follicle diameter is shown in Table 2. It was also observed (from the relation cumulus typing-nuclear progression) that from the COCs classified as type II at the time of retrieval from post-hCG patients (51.2%), 77.6% of them could remain arrested at the GV-stage for 48 hours culture. This shows that the inhibitor was effective even on the oocytes with a more expanded cumulus morphology at the time of oocyte retrieval.

### Evaluation at 72 Hours Culture

After 72 hours in the presence of DDMP, 16 out of 19 (84.2%) oocytes from pre-hCG and 10 out of 15 (66.6%) from post-hCG patients were still arrested at the GV-stage.

### Reversibility After Arrest for 48 or 72 Hours.

After 48 hours culture in DDMP, a total of 22 GV-stage oocytes were mechanically denuded from cumulus cells. Inhibitor was washed out (11 from pre-hCG and 11 from post-hCG group) and oocytes were further cultured for 24-30 h in control medium. From the pre-hCG patients group, 4 progressed to GVBD but 1 arrested at this stage and 3 of them extruded the polar body at 24-30 h. From the post-hCG patients group, 11 oocytes progressed to GVBD but 5 arrested at this stage and 6 extruded the polar body (Table 3).

TABLE 3
Number of oocytes at different stages of meiotic progression
(GV, GVBD, PB) as observed at 24-30 hours after removal of DDMP
from the maturation medium. Data grouped by duration of culture days
in DDMP supplemented medium and by type of patient
treatment (hCG or not)

| Day of | | | Oocyte stage 24-30 hours | | |
|---|---|---|---|---|---|
| Inhibitor | Patient | Number of | after DDMP removal | | |
| removal | treatment | oocytes | GV | GVBD | PB (%) |
| 2 | pre-hCG | 11 | 7 | 1 | 3 (27)^{a} |
| | post-hCG | 11 | — | 5 | 6 (54)^{c} |
| 3 | pre-hCG | 13 | 6 | 1 | 6 (46)^{a} |
| | post-hCG | 10 | — | — | 10 (100)^{b d} |

^{a}and ^{b}denotes a significant difference by Fisher's exact test (p < 0.01).
^{c}and ^{d}denotes a significant difference by χ2 test (p < 0.05).

After 72 hours in the presence of DDMP, 13 oocytes from pre-hCG and 10 from post-hCG patients were denuded and inhibitor was washed out. After 24 hours to 30 hours culture in conditioned medium, from the pre-hCG patients group, 7 progressed to GVBD but 1 arrested at this stage and 5 extruded a PB. From the post-hCG patient group, 10 oocytes achieved PB extrusion 24-30 h after inhibitor removal (Table 3) which was significantly higher compared to the pre-hCG group (p<0.05). The proportion of oocytes with PB extrusion after inhibitor removal from the post-hCG group was also significantly higher when oocytes were cultured for 72 hours in DDMP culture compared to 48 hours (p<0.05).

### Light and Electron Microscopy

In order to analyse the effect of PDE 3 inhibition on nuclear and cytoplasm organization, ultrastructural observation was performed at the different culture times in oocytes arrested or not by DDMP. The oocytes analyzed did originate from follicles with diameter between 8-12 mm. Numbers and conditions of oocytes analysed by EM are listed in Table 1.

TABLE 1
Number of oocytes analysed by EM in the different maturation stages
(GV, GVBD, PB) at time of retrieval and 24, 48 and 72 hours after
culture. Oocytes analyzed are grouped by type of patient treatment and
culture conditions

| | | | Number of oocytes analyzed at | | | | | | |
|---|---|---|---|---|---|---|---|---|---|
| | | | different times of culture in the | | | | | | |
| | | | different stages of nuclear maturity | | | | | | |
| | Number of | | | | | | | | After 72 |
| | oocytes at | Culture | After 24 | | | After 48 | | | Hours |
| Patient | time of re- | condi- | hours culture | | | hours culture | | | culture |
| treatment | trieval | tions | GV | GVBD | PB | GV | GVBD | PB | GV |
| Pre-hCG | 2 | Control | | 2 | 4 | | | 9 | |
| | | Inhibitor | 3 | | | 4 | | 1 | 3 |
| Post-hCG | 2 | Control | 2 | 1 | 4 | | 1 | 2 | |
| | | Inhibitor | 3 | 1 | 1 | 3 | | | |
| Total | 4 | | 8 | 4 | 9 | 7 | 1 | 12 | 3 |

### Oocyte Morphology at Time of Retrieval-0 Hour

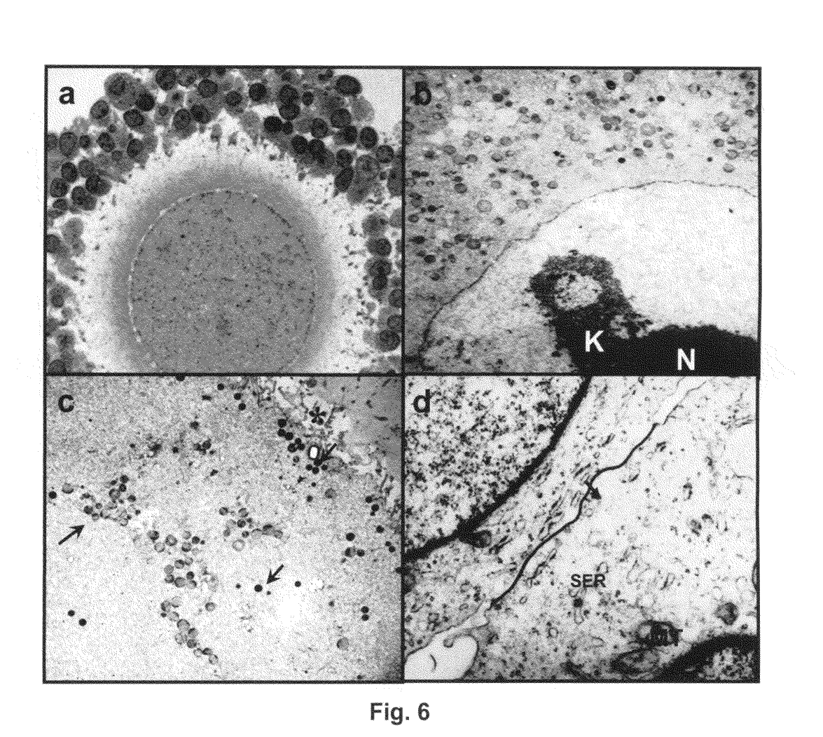

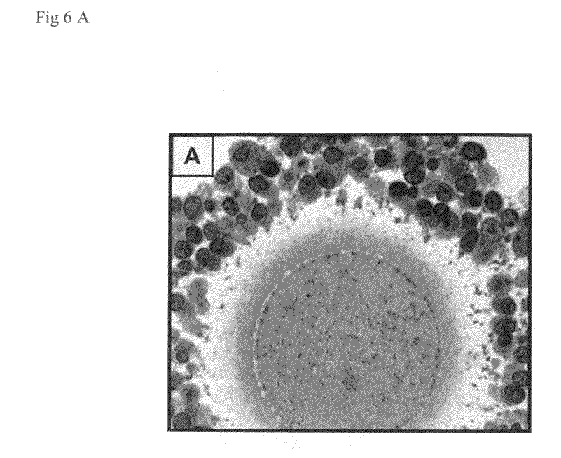

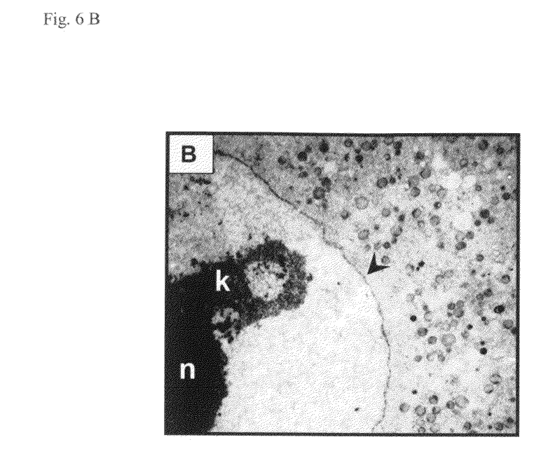

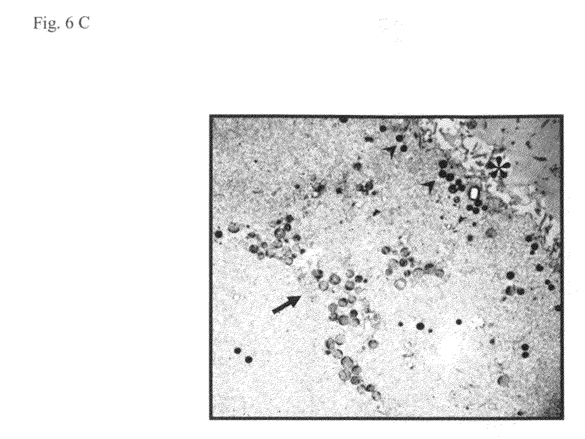

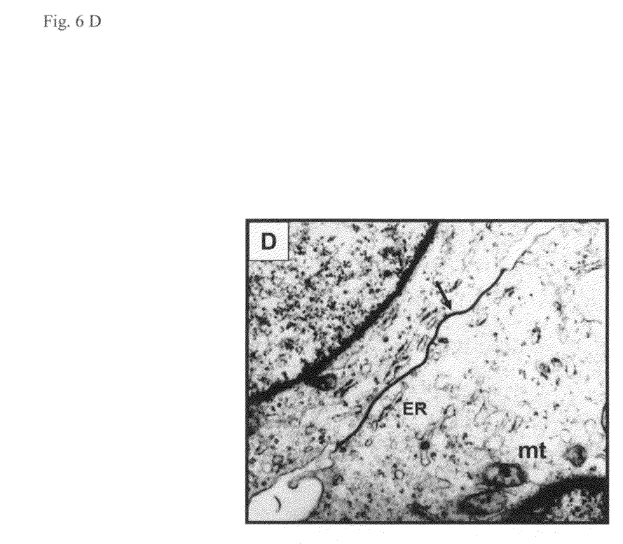

Semithin and ultrathin sections revealed that the GV of these oocytes was located centrally (three oocytes) or close to the plasmalemma (in one oocyte). The nucleus consisted of an envelope formed by two layers of membrane, containing one dense compact nucleolus associated with inter-chromatin granular complexes and extranucleolar chromatoid bodies on the periphery of nucleoli (Sathananthan, 1985). The nucleoli are regularly shaped, round or oval, homogeneous and formed by fibrillar granules. In all oocytes analysed the formation of nucleolus-associated chromatin was observed as a continuous mass (karyosphere), which normally exists in oocytes obtained from antral follicles of 8 mm and larger (Tesarik et al, 1983). The karyosphere, also called as surrounded nucleolus (SN), occupied an excentric position in the nucleus and consists of loosely packed fibrills in contact to the nucleolus (FIG. 6b). The presence of karyosphere was independent of the position of the nucleus in the oocyte (only in one oocyte the nucleus was at the periphery).

At the level of organization of the cytoplasm, a characteristic of all the oocytes analyzed at retrieval was the presence of areas devoid of organelles at the cortex and intermediate cytoplasm. Clumps of mitochondriae were found distributed throughout the cytoplasm (FIG. 6a,c). At the surrounding of the GV aggregates of mitochondria were intermingled with vesicles of different diameter. Cortical granules were found dispersed throughout the cytoplasm and few were observed under the oolemma of the oocyte forming a single layer.

Microvilli were noticed extending from the plasma membrane through the inner cortex of the zona pellucida (ZP). Numerous cell projections penetrated the zona pellucida to the oolemma. The granulosa cells (GC) appeared as oval or round cells apposed to the ZP with numerous cellular projections crossing the ZP reaching the oolemma. Cells displayed a compact arrangement and tightly adhered to each other by closely apposed plasma membranes resembling gap junctions (FIG. 6d). The nuclei of the cells are excentrically located. Granulosa cells of the COC did not show signs of pyknosis.

Morphology of COCs after 24 Hours Culture without Inhibitor

Electron microscopy analyses of oocytes cultured in control medium demonstrated that in vitro maturation was associated with reorganization of mitochondria and smooth endoplasmic reticulum (SER) which appear as round vesicles into cytoplasm. This reorganization was not different for oocytes retrieved pre or post-hCG stimulation. Mitochondriae are spherical or oval and are dispersed throughout the cytoplasm or around aggregates of SER. These aggregates form characteristic complexes with peripheral mitochondria in the maturing human oocytes (vide Ann. NY Acad. Sci. 442 (1986), 251 et seq. and Prog. Clin. Biol. Res. 296 (1989), 273 et seq.) and were predominantly present in the PB-extruded ones.

Cortical granules (CG) showed a discontinuous distribution on the surface of the GVBD oocytes and appeared in one to two layers at the cortex of PB-extruded oocytes.

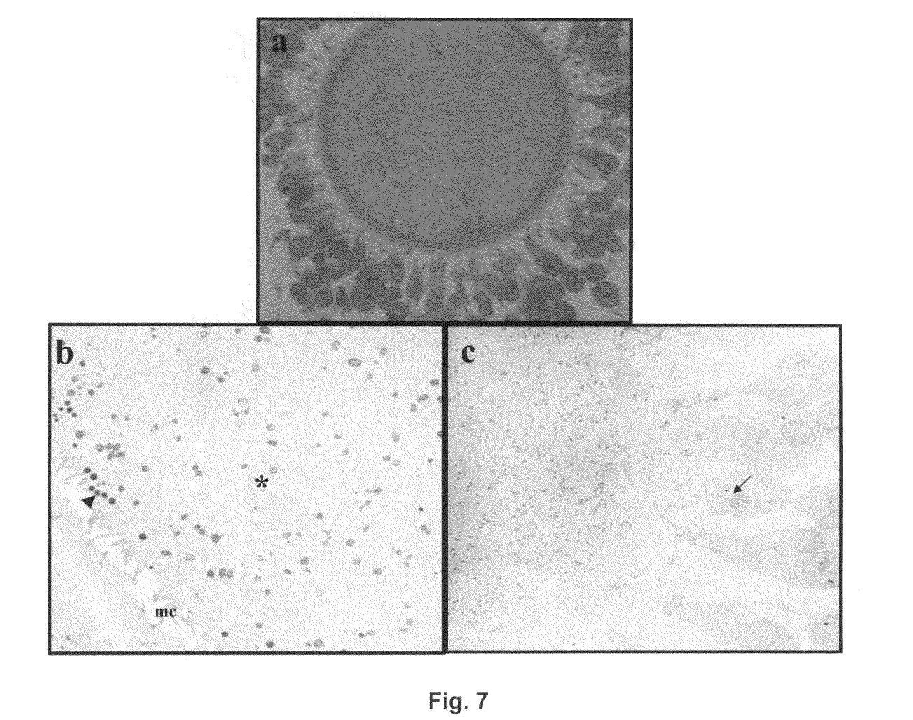

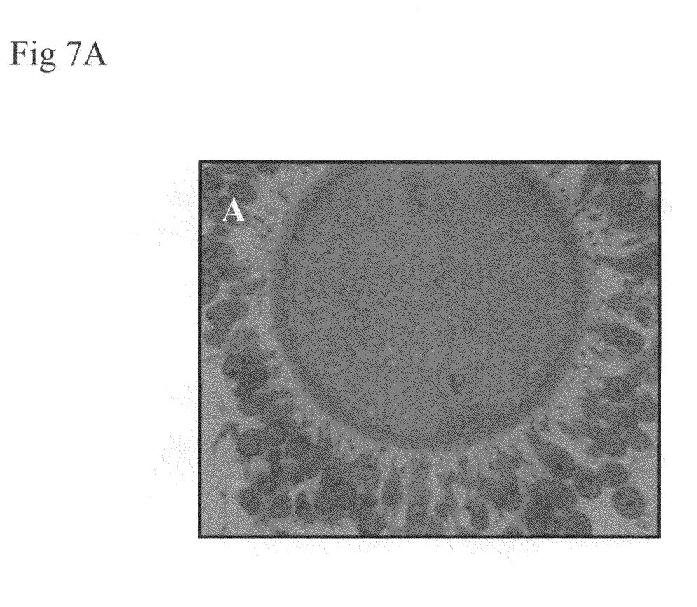

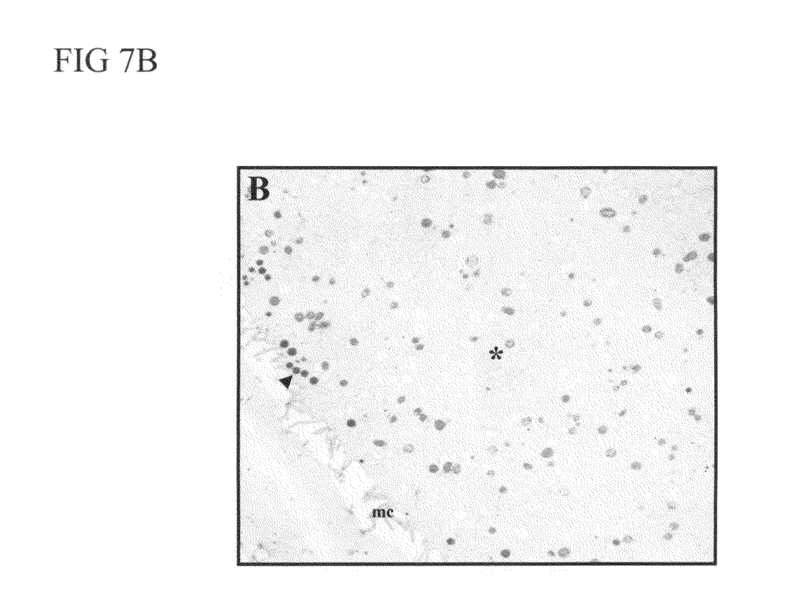

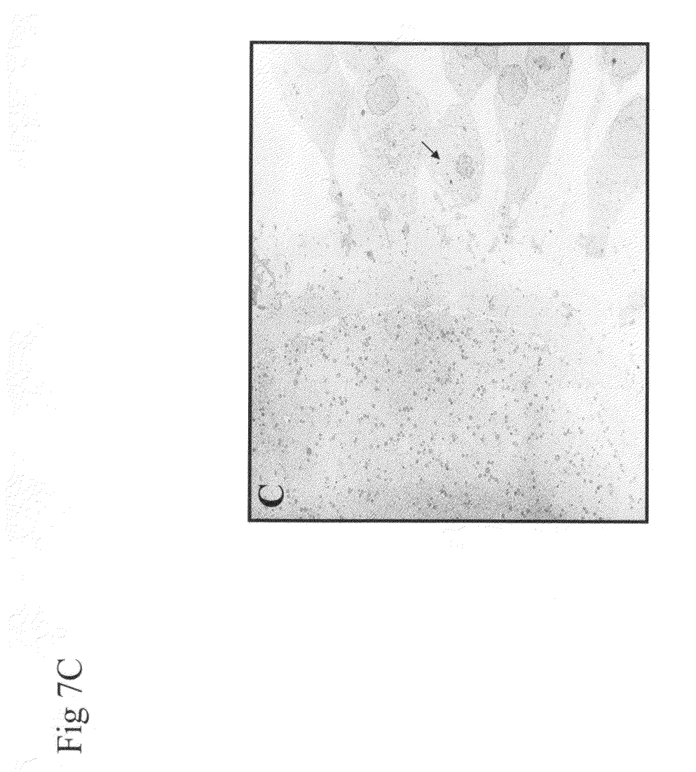

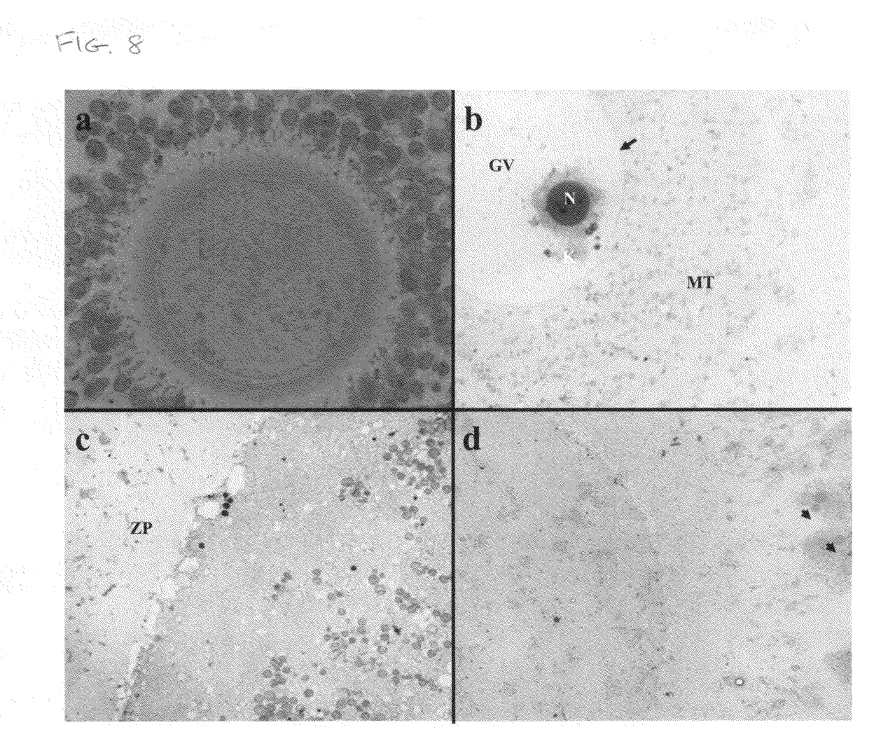

The oocyte that remained at the GV-stage after 24 hours in control medium had the same type of nucleus (with presence of karysphere) and organelle distribution as for the GVs fixed and analyzed at time of retrieval (FIG. 7b).

After 24 hours of culture cumulus cells were more elongated, retracted from the zona pellucida and partially detached from the oocyte. The nuclei of the cells were displaced to the periphery of the cell at the opposite side of the elongation (FIG. 7a, c).

Morphology of COC After 24 Hours Culture with Inhibitor

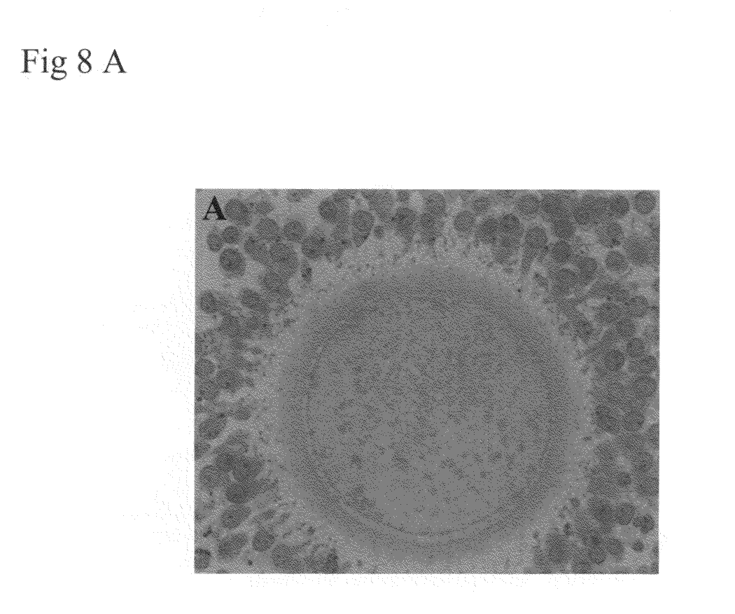

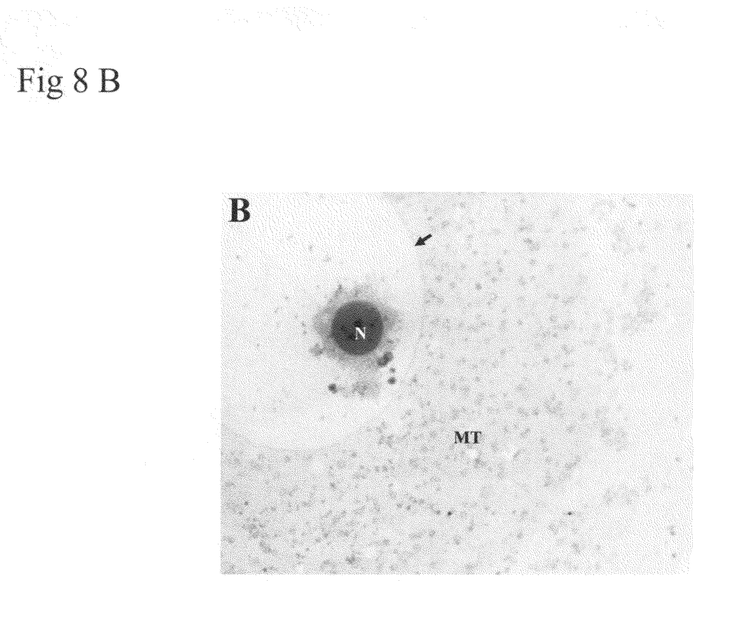

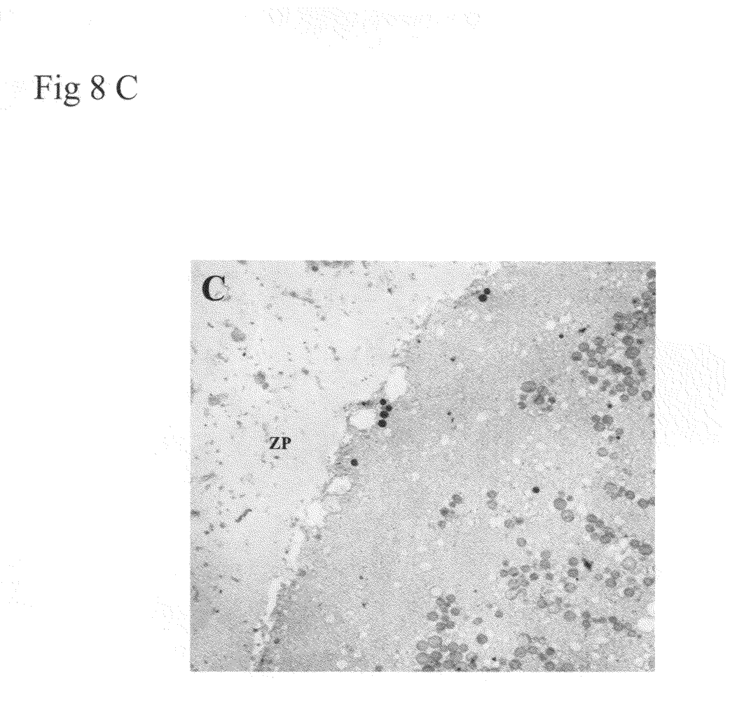

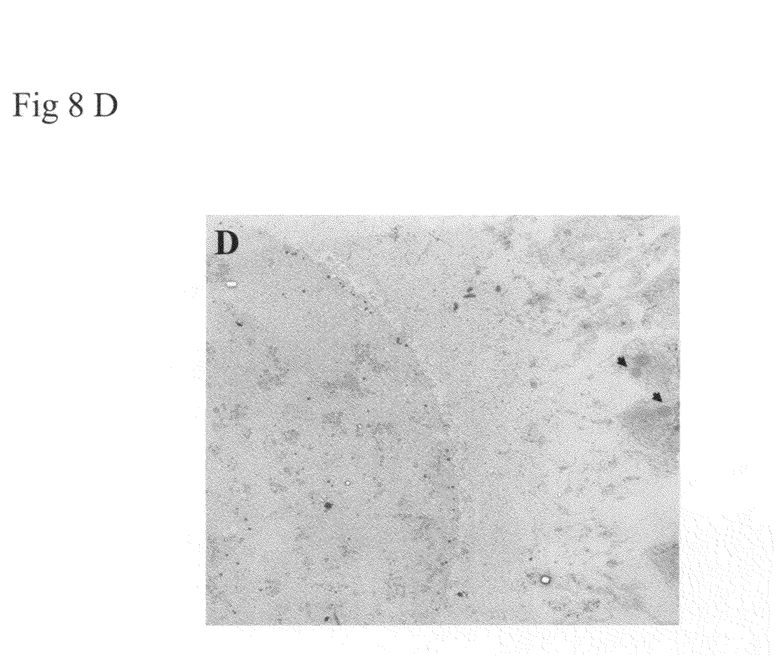

The GV of the oocytes were centrally located with presence of a karyosphere around the nucleoli of all oocytes. The nuclear membranes of all oocytes had remained intact (FIG. 8b).

All oocytes showed signs of immature cytoplasm similar to the ones fixed at time of retrieval. Mostly, the mitochondriae were found distributed in clumps throughout the cytoplasm (FIG. 8c). In the surroundings of the GV, aggregates of mitochondria were intermingled with vesicles of different diameter. Few cortical granules were found dispersed throughout the cytoplasm and beneath the oolemma of the oocyte, forming a single layer. A single GV-stage oocyte (retrieved from a 9 mm post-hCG follicle aspiration) had no presence of karyosphere in the nucleus and a more homogeneous distribution of organelles in the cytoplasm. The cytoplasm of the GVBD oocyte analysed showed an inhibition of the distribution of organelles, which were aggregated as in the cytoplasm of a GV-stage oocyte. The oocyte that underwent PB extrusion had the same cytoplasmic morphology as the control ones.

The cumulus cells started to expand and loose contact with the oocyte. Cumulus cells started their elongation, although some cells were still round or oval. The nuclei of these cells did not yet migrate to the periphery (FIG. 8a).

Morphology of COC After 48 Hours Culture without Inhibitor

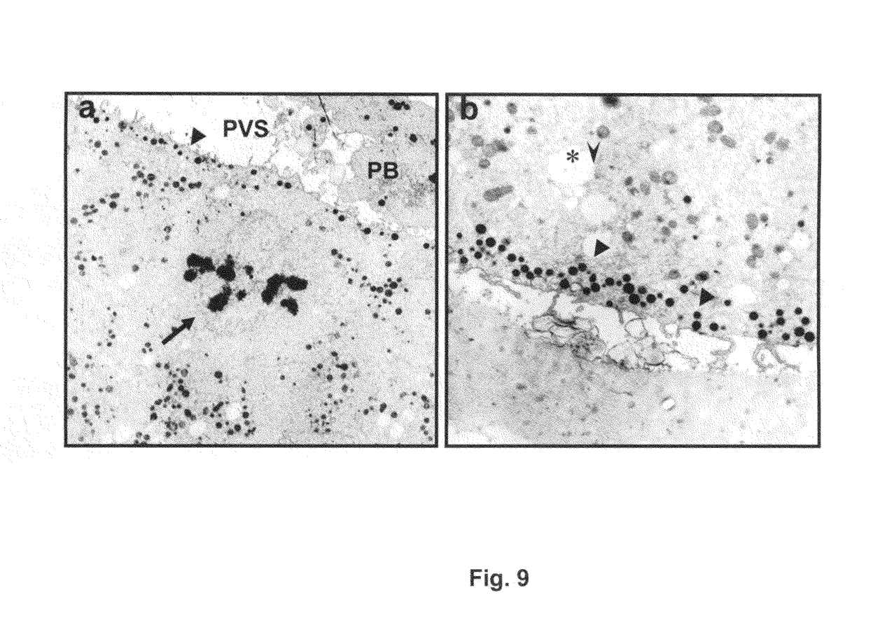

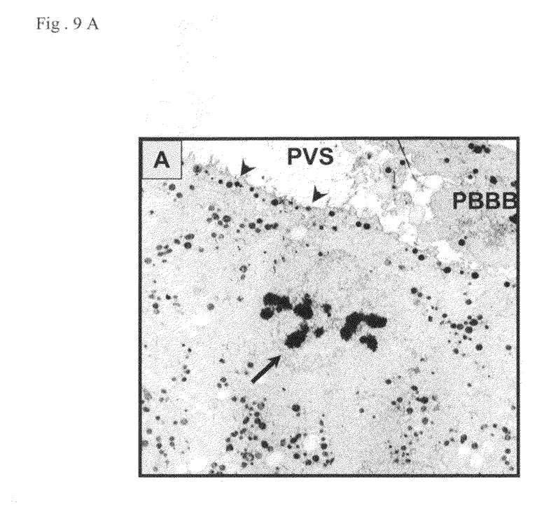

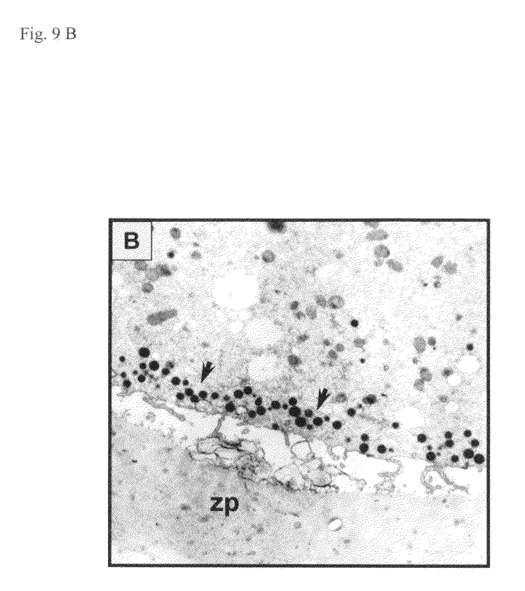

The PB-extruded oocytes analysed after 48 hours culture in controls formed the meiotic spindle perpendicular to the oolemma (FIG. 9a). The organelles, mitochondriae and SER were spread throughout the ooplasm as vesicles or as aggregates of tubular elements. The CG formed one to three layers under the oolema, and in one matured oocyte (pre-hCG), numerous CG were conglomerated beneath the oolema at the place of the meiotic spindle. Some swollen SER vesicles were observed perhaps as a sign of oocyte ageing (Sathananthan, 1982) (FIG. 9b). In all oocytes few CG could also be observed throughout the cytoplasm. An increase of vesiculation was not observed in any of these oocytes.

Most oocytes had their surrounding cells more dispersed or completely detached from the zona and less transzonal processes appeared from cumulus cells into the ZP.

Morphology of COC After 48 Hours and 72 Hours Culture with Inhibitor

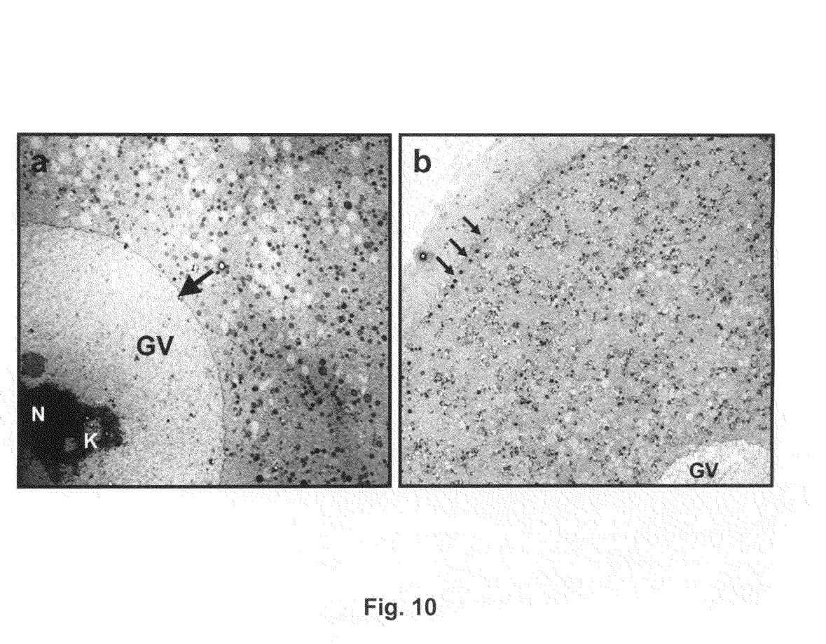

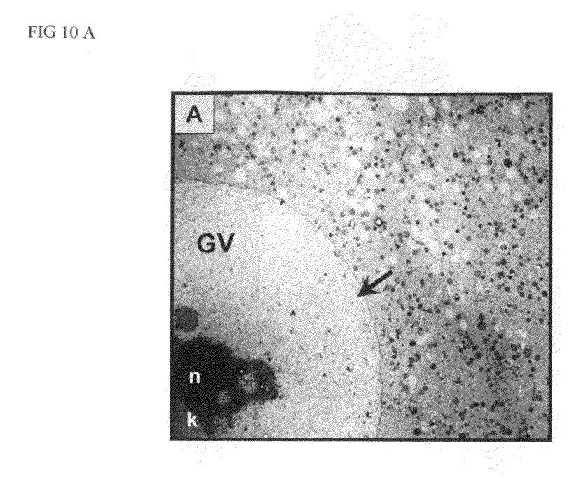

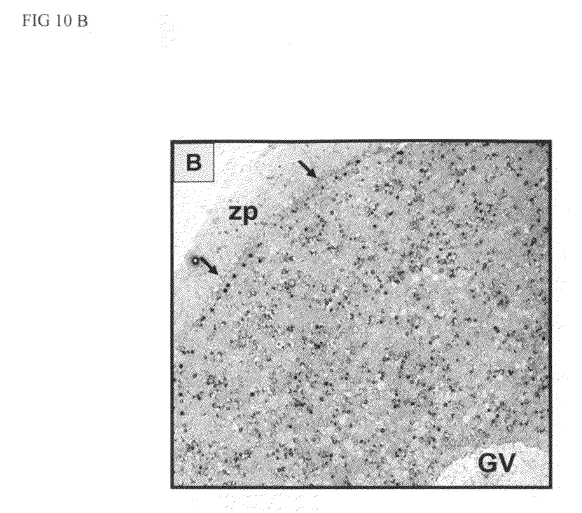

The GV-stage oocytes fixed and analyzed after 48 hours and 72 hours culture in the presence of the inhibitor showed a centrally located nucleus with presence of karyosphere around the nucleoli in almost all oocytes (FIG. 10a). One GV-stage oocyte at 48 hours (retrieved from one 8 mm pré-hCG follicle aspiration) had no presence of karyosphere in the nucleus and a more homogeneous distribution of organelles in the cytoplasm. The nuclear membranes of all oocytes had remained intact. The organelles were still aggregated, although in some oocytes the mitochondria started to dissociate from their aggregates. Few cortical granules were found dispersed throughout the cytoplasm and under the oolemma of the oocyte forming a single layer (FIG. 10b). The cumulus cells were more dispersed or completely detached from zona with less transzonal processes through the ZP. The oocytes that underwent maturation (GVBD and PB) had the same cytoplasmic morphology as the controls.

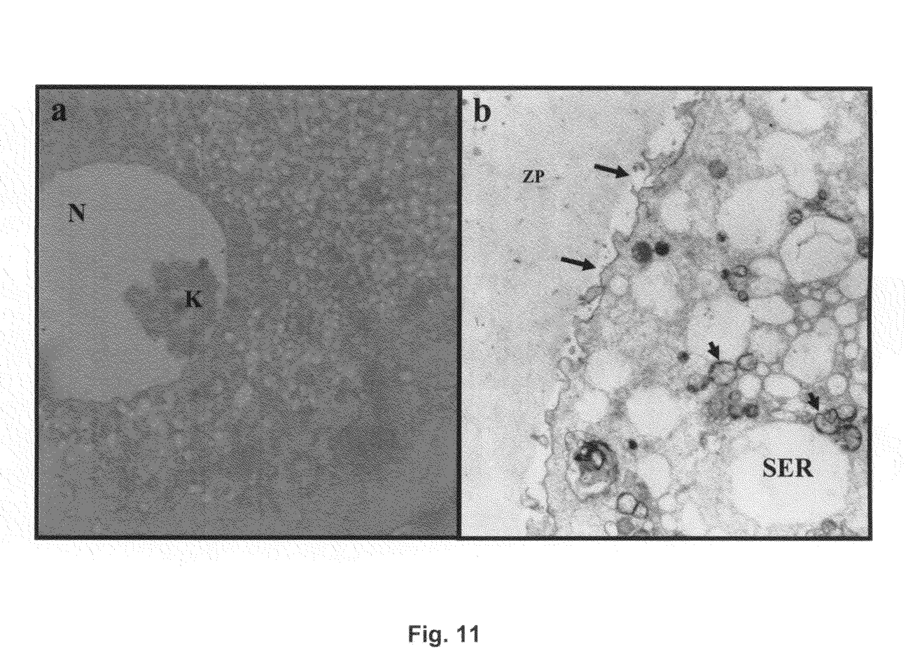

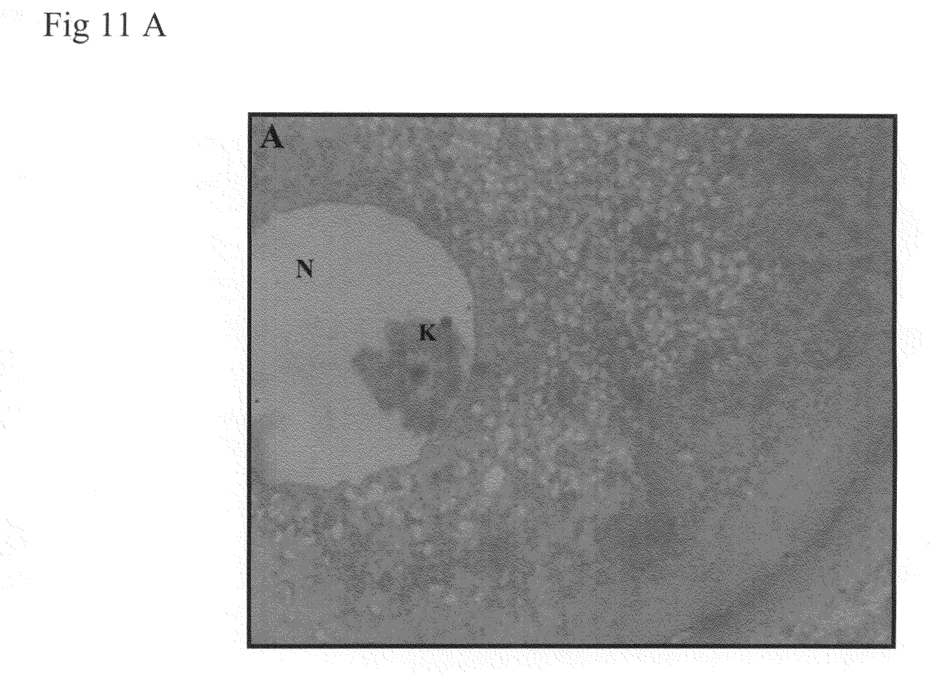

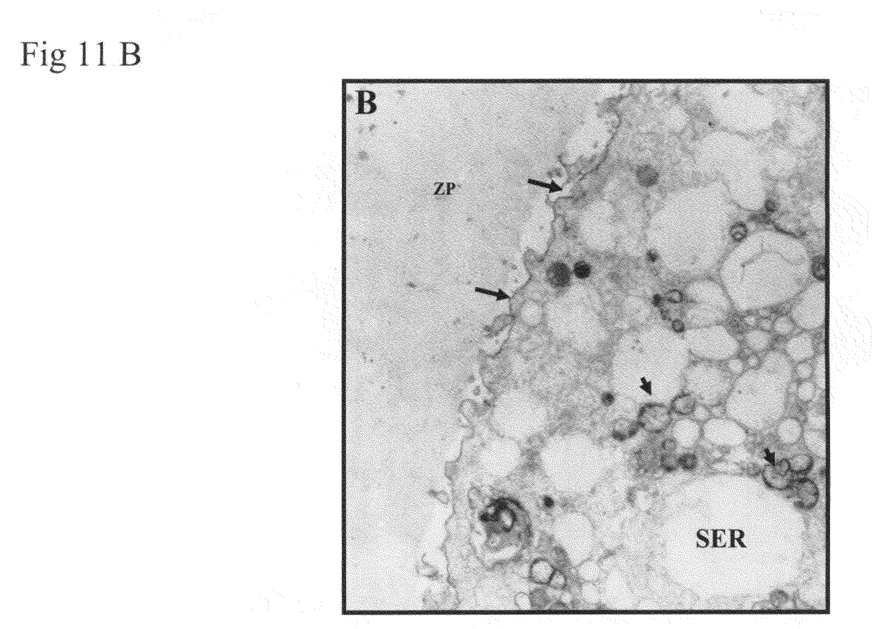

After 72 hours, the oocytes showed signs of vacuolization in the ooplasm, resulting from swelling of SER (FIG. 11a, b). Microvilli were less evident and zona was more homogeneous with no transzonal processes from cumulus cells, indicating a complete loss of connections with the oocyte (FIG. 11b).

### Confocal Laser Microscopy for Microfilaments and Chromosome Analysis

A total of twenty oocytes (7 out of 8 pré-hCG; and 13 out of 17 post-hCG) which were stained after 48 hours culture in control medium presented a metaphase II (MII) plate of which 80.0% had well-aligned chromosomes and positioned perpendicular to the oolema. One oocyte presented chromosomes dislocated from the M II plate (FIG. 12). Four oocytes had nuclei forming a clump of chromosomes at the place of metaphase plate, probably as a sign of ageing. Microfilaments were observed in the scanned levels of oocytes with a more prominent staining at the optical sections close to the site of first polar body extrusion described as generalized pattern (Terada et al, 1995). No superposed microfilaments of actin were observed at the site of polar body extrusion, showing a homogeneous staining at this level.

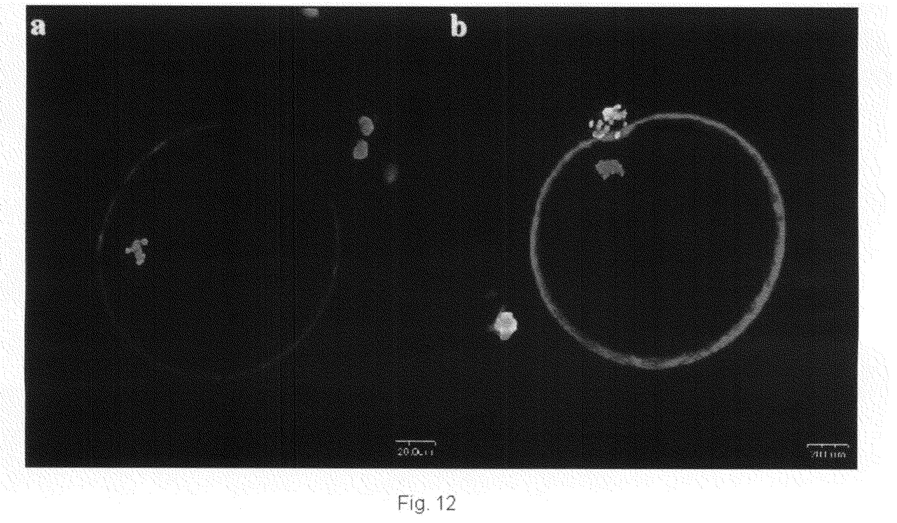

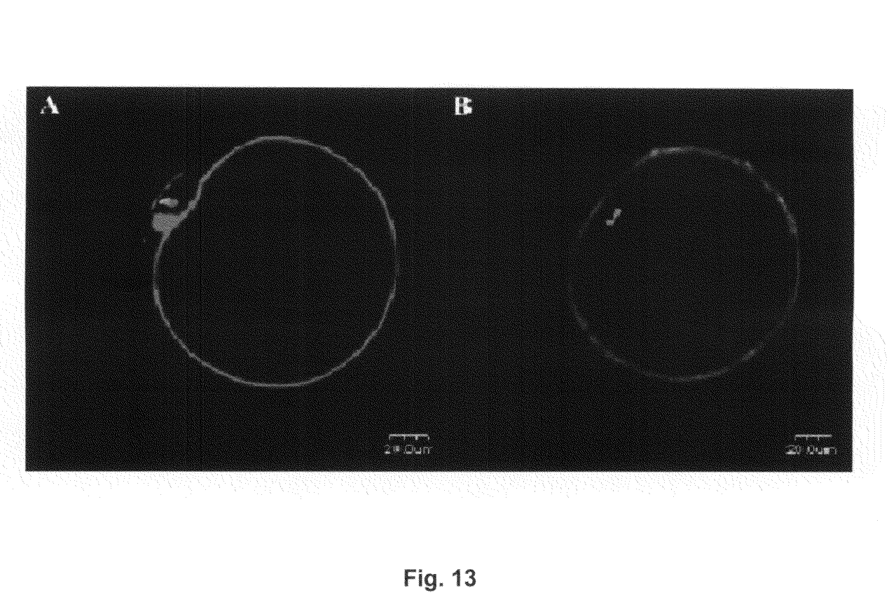

Twelve oocytes presented the M II plate 24-30 hours after inhibitor removal (3 out of 3 pre-hCG, and 9 out of 11 post-hCG) in which 83.3% had were well-aligned chromosomes and positioned perpendicular to the oolema (FIG. 13). Two oocytes had nuclei forming a clump of chromosomes at place of metaphase plate. The generalized aspect of microfilaments was also observed in those oocytes with homogeneous prominent staining at the level of the polar body extrusion site (FIG. 13).

Use of DDMP allowed demonstrating that PDE3 significantly participates in the regulation of human oocyte maturation. Used at a dose of 10 μM DDMP consistently blocked resumption of meiosis in vitro in COCs extracted from antral follicles for at least 48 hours culture without ultrastructural morphological signs of oocyte degeneration. Nuclear arrest could be held on up to 72 hours of culture. After inhibitor removal, the arrested oocytes were capable of resuming meiosis within the normal time frame.

We experienced that, by using a selective DDMP, meiotic arrest in GV could be maintained effectively for a period of at least 48 hours. It was also observed that, in this serum-free culture medium, cumulus cells start to loose connections to the oocyte after 24 hours. In oocytes collected after hCG pre-treatment, breakdown of connections is even more rapid and transzonal processes can be kept only for a maximal period of about 48 hours. The DDMP itself had no influence on this progressive loss of coupling between somatic cells and oocyte. This emphasises the advantage of using a selective inhibitor acting directly into the oocyte cAMP metabolism via the intra oocyte PDE3A bypassing the needs for somatic cell-dependent cAMP generation to arrest the nuclear maturation.

Using a selective DDMP to block cAMP degradation in the oocyte produced normal morphological signs of nuclear stagnation of GV oocytes for 72 hours. The nuclear membrane remained intact and unfolded, the surrounding nucleus chromatin configuration (SN or karyosphere) was persistent during arrest and the germinal vesicle was most often located in a central position. It has been reported that karyosphere formation reflects the state of oocyte nucleus being prepared for ovulation with extinction of transcriptional processes. The karyosphere was detectable in the nucleus of the oocytes when they were still surrounded by a compact cumulus cell mass. Several transzonal processes onto the oocytes were observed with a heterogeneous zona and microvilli were still present and abundant.

Prolonged nuclear arrest by DDMP in our in vitro maturation medium seemed to affect the migration of organelles in the GV-stage oocytes. The organelles start to dissociate from the aggregates after 48 hours in DDMP culture, even in GV-intact oocytes. In the DDMP arrested oocytes, cortical granules had formed a characteristic single layer, while in contrast controls had their cortical granules still conglomerated.

Studies regarding the use of DDMP on oocytes were done in rodents; it was confirmed that blocking effects are fully reversible in isolated oocytes. After pharmacological arrest of human oocytes for 48 or 72 hours, the meiotic cycle can be reinitiated by washing out the DDMP. Depending on follicle pretreatment conditions (HMG stimulation alone or in combination with an HCG stimulation), 46% to 100% of oocytes could normally progress through meiosis with formation of a metaphase II plate with fidelity of chromosome segregation.

### EXAMPLE 2

The evaluation of whether a specific compound is a PDE31NH or not can, for example, be made using the following test:

An example of such an experiment (i.e. to establish the specificity and dose dependent ability of PDE3, but not PDE4, inhibitors to block the spontaneous maturation of meiosis) was described by Jensen et al. (vide Human Reproduction 17 (2002), 2019-2084.

### EXAMPLE 3

Herein, the term MAS compound designates compounds which mediate the meiosis of oocytes. More specifically, a MAS compound is a compound which in the test described below in this example has a percentage germinal vesicle breakdown (hereinafter designated GVB) which is significantly higher than the control. Preferred MAS compounds are such having a percentage GVB of at least 50%, preferably at least 80%.

Examples of MAS compounds are mentioned in WO 96/00235, 96/27658, 97/00884, 98/28323, 98/54965 and 98/55498, more specifically in Claim 1 thereof.

The evaluation of whether a specific compound is a MAS compound or not can, for example, be made using the following test:

Oocytes were obtained from immature female mice (C57BU6J×DBA/2J F1, Bomholtgaard, Denmark) weighing 13-16 grams, that were kept under controlled temperature (20-22° C.), light (lights on 06.00-18.00) and relative humidity (50-70%). The mice received an intraperitoneal injection of 0.2 ml gonadotropins (Gonal-F, Serono) containing 20 IU FSH and 48 hours later the animals were killed by cervical dislocation. The ovaries were dissected out and the oocytes were isolated in Hx-medium (see below) under a stereomicroscope by manual rupture of the follicles using a pair of 27 gauge needles. Spherical oocytes displaying an intact germinal vesicle (hereinafter designated GV) were divided in cumulus enclosed oocytes (hereinafter designated CEO) and naked oocytes (hereinafter designated NO) and placed in α-minimum essential medium (α-MEM without ribonucleosides, Gibco BRL, Cat. No. 22561) supplemented with 3 mg/ml bovine serum albumin (BSA, Sigma Cat. No. A-7030), 5 mg/ml human serum albumin (HSA, Statens Seruminstitut, Denmark), 0.23 mM pyruvate (Sigma, Cat. No S-8636), 2 mM glutamine (Flow Cat. No. 16-801), 100 IU/ml penicillin and 100 μg/ml streptomycin (Flow, Cat No. 16-700). This medium was supplemented with 3 mM hypoxanthine (Sigma Cat. No. H-9377) and designated Hx-medium. The oocytes were rinsed three times in Hx-medium and oocytes of uniform size were divided into groups of CEO and NO. CEO and NO were cultured in 4-well multidishes (Nunclon, Denmark) in which each well contained 0.4 ml of Hx-medium and the compound to be tested in a concentration of 10 μM. One control well (i.e., 35-45 oocytes cultured in identical medium with no addition of test compound) was always cultured simultaneously with 3 test wells (35-45 oocytes per well supplemented with test compound). The oocytes were cultured in a humidified atmosphere of 5% CO₂ in air for 24 hours at 37° C. By the end of the culture period, the number of oocytes with GV, GVB and polar bodies (hereinafter designated PB), respectively, were counted using a stereo microscope (Wildt, Leica MZ 12). The percentage of GVB, defined as percentage of oocytes undergoing GVB per total number of oocytes in that well, was calculated as:

% GVB=((number of GVB+number of PB)/total number of oocytes)×100.

What we claim is:

1. A method for in vitro synchronisation of nuclear and cytoplasmatic maturation of GV oocytes from domestic animals or from primates comprising the steps of:
a. culturing one or more GV oocytes or Ml oocytes from domestic animals or from humans in a first culture medium, the culture medium comprising a pyridazinone compound of formula I, 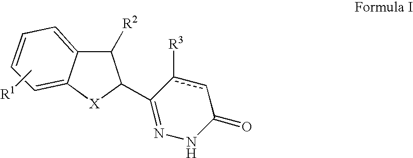
wherein R1 represents one to four substituents, which may be the same or different and are selected from H, OH, halogen, NO2, unsubstituted or C1-C4 alkyl substituted amino, C1-C4 alkyl, C1-C4 halogen substituted alkyl, O-ALKNR4R5, C1-C4 alkoxy, whereby two substituents taken together may also represent a methylenedioxy group; R2 and R3 represent independently H or C1-C4 alkyl; R4 and R5 represent independently H or C1-C4 alkyl, or form together with the nitrogen a 5- or 6-membered ring; X represents S or O; the dotted line represents an optional bond; and their pharmaceutically acceptable salts, the culturing taking place for a time period sufficient for cytoplasmatic maturation to progress or to allow for normal working hours during the IVF/ICSI procedure, the culturing taking place for a time period sufficient for cytoplasmatic maturation to progress or to allow for normal working hours during the IVF/ICSI procedure;
b. washing the oocytes of step (a) to remove the pyridazinone compound of formula I or decrease the amount thereof;
c. culturing the washed oocytes of step (b) in a second culture medium for a time period sufficient for nuclear maturation to be completed up to metaphase II to a desired frequency.

2. The method according to claim 1, wherein human oocytes are used.

3. The method according to claim 2, wherein the oocytes used for culturing in the first medium are oocytes which have been stored in an oocyte collection medium, wherein the oocyte collection medium is a CO₂-independent medium containing the pyridazinone compound of formula I.

4. The method according to claim 3, wherein the oocyte has been placed in said oocyte collection medium not later than 4 hours.

5. The method according to claim 1, wherein the culturing according to step (a) takes place for a time period sufficient for cytoplasmatic maturation to be improved.

6. The method according to claim 1, wherein the culturing according to step (a) takes place for a time period sufficient for cytoplasmatic maturation to be completed.

7. The method according to claim 1, wherein the oocytes cultured in step (a) are GV oocytes.

8. The method according to claim 1, wherein the first culture medium contains EGF, GH, gonadotropin combinations, promoters of glutathione synthesis, meiosis activating compounds, combinations of activin and inhibin, the analogues of the foregoing or combinations of the aforementioned compounds.

9. The method according to claim 1, wherein the pyridazinone compound of formula I is DDMP.

10. The method according to claim 1, wherein the culturing time in step (a) is at least about 4 hours.

11. The method according to claim 1, wherein the culturing time is at least about 8 hours.

12. The method according to claim 1, wherein the culturing time is at least about 12 hours.

13. The method according to claim 1, wherein the culturing time is at least about 24 hours.

14. The method according to claim 1, wherein the culturing time is at least about 48 hours.

15. The method according to claim 1, wherein the culturing time is at least about 72 hours.

16. The method according to claim 1, wherein the oocytes used in step (a) are collected from follicles which were aspirated from the animal/human before any hormone treatment that could effect either folliculogenesis and/or oogenesis)

17. The method according to claim 16, wherein the hormone treatment of the woman from which the oocytes originates were performed with a steroid, a gonadotrophin, a GnRHanalogue, clomiphene citrate, tamoxiphen, an insulin sensitiser (e.g. metformin), an aromatase inhibitor or any of the preceding drugs alone or combined.

18. The method according to claim 1, wherein the oocytes are collected from follicles which were aspirated from the animal after any hormone or drug treatment having effects on folliculogenesis and/or oogenesis.

19. The method according to claim 18, wherein the hormone or drug treatment is performed with a gonadotropin, a GnRHanalogue, clompihene citrate, tamoxiphen, LH, HCG, a steroid or a steroid-like substance, an LHRH analogue, an aromatase inhibitor, an insulin sensitizing agent, and any analogue or combination thereof.

20. The method according to claim 18, wherein the hormone used is hCG, LH or any compound which might induce similar effects.

21. The method according to claim 1 wherein the washing of the oocytes from step (a) is performed so that the concentration of the pyridazinone compound of formula I after the washing out of the pyridazinone compound of formula I in the second medium is less than about 10% of the concentration of the pyridazinone compound of formula I in the first medium.

22. The method according to claim 1 wherein the first culture medium is minimally covered by a thin layer of an oil so that the ratio between the oily phase and the aqueous phase is less that about 2:10 (vol/vol).

23. The method according to claim 1 whereby the oocyte collection medium is covered by oil.

24. The method according to claim 1 where, in step (a), there are cumulus cells around the oocyte.

25. The method according to claim 1 where, in step (b), washing the oocytes of step (a) to remove the pyridazinone compound of formula I or decrease the amount thereof is performed so that the remaining amount of the pyridazinone compound of formula I, if any, has no substantial effect on the arresting of the oocytes.

26. The method according to claim 1 where, in step (c), there are no or substantially no cumulus cells around the oocyte.

27. The method according to claim 1, wherein, in step (c), the nuclear maturation is stimulated by adding HCG and/or EGF and/or a MAS compound to the second culture medium.

28. The method according to claim 1, wherein, in step (c), the desired frequency is at least about 30%.

29. The method according to claim 1 whereby, after step (c), the oocytes are fertilized with sperm.

30. The method according to claim 3, wherein the oocyte has been placed in said oocyte collection medium not later than 2 hours after said oocyte has been removed from the woman.

31. The method according to claim 3, wherein the oocyte has been placed in said oocyte collection medium not later than 1 hour after said oocyte has been removed from the woman.

32. The method according to claim 3, wherein the oocyte has been placed in said oocyte collection medium not later than % an hour after said oocyte has been removed from the woman.

33. The method according to claim 1 wherein the washing of the oocytes from step (a) is performed so that the concentration of the pyridazinone compound of formula I after the washing out of the pyridazinone compound of formula I in the second medium is less than about 5% of the concentration of the pyridazinone compound of formula I in the first medium.

34. The method according to claim 1 wherein the washing of the oocytes from step (a) is performed so that the concentration of the pyridazinone compound of formula I after the washing out of the pyridazinone compound of formula I in the second medium is less than about 1% of the concentration of the pyridazinone compound of formula I in the first medium.

35. The method according to claim 1 wherein the first culture medium is minimally covered by a thin layer of an oil so that the ratio between the oily phase and the aqueous phase is less that about 1:10 (vol/vol).

36. The method according to claim 1, wherein, in step (c), the desired frequency is at least about 50%.

37. The method according to claim 1, wherein, in step (c), the desired frequency is at least about 70%.

38. The method according to claim 1, wherein, in step (c), the desired frequency is at least about 90%.

39. The method according to claim 29 wherein the oocytes are fertilized using ICSI.
